(19) United States
(12) Patent Application Publication
(10) Pub. No.: US 2021/0405074 A1
(43) Pub. Date: Dec. 30, 2021

(54) BIOMARKERS FOR THE DIAGNOSIS AND CHARACTERIZATION OF ALZHEIMER'S DISEASE

(71) Applicants: Rima F. Kaddurah-Daouk, Belmont, MA (US); Jon B. Toledo, Philadelphia, PA (US); Matthias Arnold, Neuherberg (DE); Gabi Kastemüller, Neuherberg (DE); Rebecca A. Baillie, San Carlos, CA (US); Xianlin Han, Orlando, FL (US); Will Thompson, Durham, NC (US); Lisa St. John-Williams, Durham, NC (US); Therese Koal, Innsbruck (AT); Kwangsik Nho, Indianapolis, IN (US); M. Arthur Moseley, Raleigh, NC (US); Andrew J. Saykin, Indianapolis, IN (US); Pudugramam Murali Doraiswamy, Chapel Hill, NC (US)

(72) Inventors: Rima F. Kaddurah-Daouk, Belmont, MA (US); Jon B. Toledo, Philadelphia, PA (US); Matthias Arnold, Neuherberg (DE); Gabi Kastemüller, Neuherberg (DE); Rebecca A. Baillie, San Carlos, CA (US); Xianlin Han, Orlando, FL (US); Will Thompson, Durham, NC (US); Lisa St. John-Williams, Durham, NC (US); Therese Koal, Innsbruck (AT); Kwangsik Nho, Indianapolis, IN (US); M. Arthur Moseley, Raleigh, NC (US); Andrew J. Saykin, Indianapolis, IN (US); Pudugramam Murali Doraiswamy, Chapel Hill, NC (US)

(21) Appl. No.: 16/331,940

(22) PCT Filed: Sep. 8, 2017
(86) PCT No.: PCT/US2017/050831

§ 371 Date: Mar. 8, 2019

Related U.S. Application Data
(60) Provisional application No. 62/384,854, filed on Sep. 8, 2016.

(51) Int. Cl. G01N 33/92

(52) U.S. Cl. CPC G01N 33/92; G01N 2800/50; G01N 2800/52; G01N 2800/2821

(57) ABSTRACT

Embodiments of the present disclosure relate generally to the analysis and identification of global metabolic changes in Alzheimer's disease (AD). More particularly, the present disclosure provides materials and methods relating to the use of metabolomics as a biochemical approach to identify peripheral metabolic changes in AD patients and correlate them to cerebrospinal fluid pathology markers, imaging features, and cognitive performance. Defining metabolic changes during AD disease trajectory and their relationship to clinical phenotypes provides a powerful roadmap for drug and biomarker discovery.

### CROSS-REFERENCE TO RELATED APPLICATIONS

This application is a U.S. national phase application under 35 U.S.C. § 371 of International Patent Application No. PCT/US2017/050831, filed Sep. 8, 2017, which claims priority to U.S. Provisional Patent Application Ser. No. 62/384,854, filed Sep. 8, 2016, each of which is incorporated by reference herein in its entirety.

### GOVERNMENT FUNDING

The subject matter of this invention was made with Government support under Federal Grant Nos. R01AG046171, RF1AG051550, 3U01AG024904-0954, P50NS053488, R01AG19771, P30AG10133, and P30AG10124 awarded by the National Institutes on Aging (NIA); Federal Grant Nos. RO1LM011360 and R00LM011384 awarded by the by National Library of Medicine (NLM); Federal Grant No. U01AG024904 awarded by the National Institutes of Health (NIH); and Federal Grant No. W81XWH-12-2-0012 awarded by the Department of Defense. The Government has certain rights to this invention.

### FIELD

Embodiments of the present disclosure relate generally to the analysis and identification of global metabolic changes in Alzheimer's disease (AD). More particularly, the present disclosure provides materials and methods relating to the use of metabolomics as a biochemical approach to identify peripheral metabolic changes in AD patients and correlate them to cerebrospinal fluid pathology markers, imaging features, and cognitive performance.

### BACKGROUND

The advent of Alzheimer's disease (AD) is the most common cause of dementia. An anticipated 136 million people will be affected by dementia by 2050, presenting major global health and economic challenges. There are currently no treatments that modify AD; hence, AD remains the largest unmet medical need within neurological disorders.

Many biochemical processes are affected in AD, including amyloid precursor protein metabolism, phosphorylation of tau protein, oxidative stress, impaired energetics, mitochondrial dysfunction, inflammation, membrane lipid dysregulation, and neurotransmitter pathway disruption. Impaired cerebral glucose uptake occurs decades before the onset of cognitive dysfunction in AD, and neurotoxicity associated with AR is thought to participate in impaired neuronal energetics including mitochondrial dysfunction and release of reactive oxygen species. Growing evidence supports the concept that insulin resistance can contribute to AD pathogenesis; and therefore, AD could be regarded as a metabolic disease mediated in part by brain insulin and insulin-like growth factor resistance. Mapping the trajectory of biochemical changes in AD is therefore becoming a priority as filling knowledge gaps about disease mechanisms and their link to metabolic processes can lead to developing much-needed biomarkers and therapies.

Metabolomics provides powerful tools for mapping global biochemical changes in disease and treatment. In contrast to classical biochemical approaches that focus on single metabolites or reactions, metabolomics and lipidomics approaches simultaneously identify and quantify hundreds to thousands of metabolites. Measurement of large numbers of metabolites enables network analysis approaches and provides means to identify critical metabolic drivers in disease pathophysiology. Initial small-scale metabolomics studies in AD have highlighted metabolic alterations including ceramide-sphingomyelin pathways, glycero-phosphatidylcholines, PE plasmalogens, amines, and mitochondrial defects among others. Metabolic networks have linked central perturbations in norepinephrine and purines with elevated cerebrospinal fluid (CSF) tau, and changes in tryptophan and methionine to decreased Ab levels.

Earlier metabolomics studies had major limitations, including not accounting for important confounds such as impact of medications use; small studies that lacked evaluation across data sets; limited ability to connect peripheral metabolic changes with central changes to define what might be related; and lack of attempts to connect metabolic changes within a pathway and network context. Network biology and “network medicine” approaches have become important tools to dissect molecular mechanisms triggering neurodegeneration. This approach accounts for the fact that complex diseases arise from alterations in multiple genes, proteins, and metabolites, and a network may be described as an interaction map among the wide range of biological entities which contribute to disease. As many of the metabolites that are associated with AD are interconnected through metabolic pathways, cofactors, and common intermediates, changes to one metabolite can entail several others, as well as have downstream effects on other co-regulated pathways. A systems biology approach integrating metabolites and their interrelations (for instance quantified by partial correlations) in metabolic networks can provide important mechanistic insights about how biochemical reactions are dysregulated during different stages of disease. In contrast to looking at single dysregulated metabolite at a time, the visualization of changes in the metabolic network captures the totality of influences on interconnected biochemical reactions in far more informative ways and allows one to follow these changes over disease stages.

### SUMMARY

Embodiments of the present disclosure provide a method for diagnosing or detecting Alzheimer's disease in a subject. In accordance with these embodiments, the method includes obtaining a sample from a subject (e.g., serum sample) and performing biochemical analysis on the sample to detect the presence of at least one biomarker metabolite. In some cases, the biomarker metabolite is a carnitine biomarker metabolite, a phosphatidylcholine biomarker metabolite, a sphingomyelin biomarker metabolite, or any combinations thereof. Detecting the biomarker metabolite can then be used to establish an association with the subject having at least one independent indicator of Alzheimer's disease, such that the subject is diagnosed with having Alzheimer's disease if at least one biomarker metabolite is detected. The method may also include administering a treatment to alleviate one or more symptoms of AD, and may also include assessing the biomarker metabolite again in order to determine if the treatment is therapeutically beneficial.

In some embodiments, the present disclosure provides methods for diagnosing or detecting Mild Cognitive Impairment (MCI) in a subject, and/or distinguishing between early phases of AD from late states of AD. In accordance with these embodiments, the method includes obtaining a sample from a subject (e.g., serum sample) and performing biochemical analysis on the sample to detect the presence of at least one biomarker metabolite. In some cases, the biomarker metabolite is a carnitine biomarker metabolite, a phosphatidylcholine biomarker metabolite, a sphingomyelin biomarker metabolite, or combinations thereof. Detecting the biomarker metabolite can then be used to establish an association with the subject having at least one independent indicator of MCI. The method may also include administering a treatment to alleviate one or more symptoms of MCI, and may also include assessing the biomarker metabolite again in order to determine if the treatment is therapeutically beneficial.

In still other embodiments, the present disclosure provides a method for predicting the outcome of a subject suspected having AD. In accordance with these embodiments, the method includes obtaining a sample from a subject and performing biochemical analysis on the sample to detect the presence of at least one biomarker metabolite. In some cases, the biomarker metabolite is a carnitine biomarker metabolite, a phosphatidylcholine biomarker metabolite, a sphingomyelin biomarker metabolite, or combinations thereof. The method may also include assessing at least one independent indicator of AD in the subject, such that detection of the at least one biomarker metabolite is associated with the subject having at least one independent indicator of AD. In some cases, the subject is predicted to develop AD if at least one biomarker metabolite is detected. The method may also include administering a treatment to alleviate one or more symptoms of MCI, and may also include assessing the biomarker metabolite again in order to determine if the treatment is therapeutically beneficial.

### BRIEF DESCRIPTION OF THE DRAWINGS

FIGS. 1A-1B include representative heat maps illustrating the clustering of pairwise metabolite correlations and association results with clinical variables. FIG. 1A is a representative heat map of Spearman correlations between the residuals of metabolite concentrations on the single metabolites. Metabolites are clustered using hierarchical clustering using the Euclidean distance metric. The clustering assigns metabolites to their biochemical class: amino acids, biogenic amines, short-chain and long-chain acylcarnitines, lysolipids, PC, and SM. Significant clusters of acyl-carnitines are outlined in blue and amines outlined in brown. FIG. 1B is a representative heat map depicting association results of the regression analyses. The distribution of association results of metabolites with clinical variables mirrors the correlation structure of the metabolites. Abbreviations: a-AAA, a-aminoadipic acid; AD, Alzheimer's disease; C0, free carnitine; Cx:y, acylcarnitines; Cx:y-OH, hydroxylacylcarnitines; Cx:y-DC, dicarboxylacylcarnitines; CN, cognitively normal; lysoPC, lyso-glycero-phosphatidylcholines (a 5 acyl); MCI, mild cognitive impairment; Path. Aβ₁₋₄₂, pathological Aβ₁₋₄₂; PC, glycero-phosphatidylcholines (aa 5 diacyl, ae 5 acyl-alkyl); SDMA, symmetric dimethylarginine; SM, sphingomyelin; SMx:y, sphingomyelins; SM (OH) x:y, N-hydroxylacyloylsphingosyl-phosphocholine; T4-OH-Pro, trans-4-hydroxyproline.

FIGS. 2A-2E include representative plots depicting the relationship between serum metabolites, clinical diagnosis, and Aβ₁₋₄₂ status. Serum PC ae 44:4 (FIG. 2A), PC ae 44:4 (FIG. 2B), and C18 (FIG. 2C) concentrations are stratified by clinical diagnosis and CSF Aβ₁₋₄₂-defined groups. The concentration of each metabolite is shown for each diagnosis red: CN, green: MCI, blue: AD and by N. Aβ: normal concentrations of Aβ₁₋₄₂ (>192 pg/mL), and Path. Aβ: pathological concentrations of Aβ₁₋₄₂ (<192 pg/mL), Y-axes are values for each metabolite. Scatter plot for ADAS-Cog13 and serum valine values are shown in FIGS. 2D and 3E. Black lines and shading represent the regression line and 95% confidence interval. Correlations between valine levels and cognitive decline in ADNI-1 and Rotterdam, respectively. Abbreviations: a-AAA, a-Aminoadipic acid; ADAS-Cog13, Alzheimer's Disease Assessment Scale-Cognition; ADNI-1, Alzheimer's Disease Neuroimaging Initiative-1; C0, free carnitine; Cx:y, acylcarnitines; Cx:y-OH, hydroxylacylcarnitines; Cx:y-DC, di-carboxylacylcarnitines; lysoPC, lyso-glycero-phosphatidylcholines (a 5 acyl); PC, glycero-phosphatidylcholines (aa 5 diacyl, ae 5 acyl-alkyl); SDMA, symmetric dimethylarginine; SMx:y, sphingomyelins; SM (OH) x:y, N-hydroxylacyloylsphingosyl-phosphocholine; T4-OH-Pro, trans-4-hydroxyproline.

FIGS. 3A-3B include representative plots of longitudinal associations for SM C20:2. FIG. 3A is a representative plot depicting Cox hazards modelling of the association of conversion from MCI to AD. Black line: 1st tertile, red line: 2nd tertile, green line: 3rd tertile. Analysis was conducted using quantitative values, and stratification by tertiles was used only for graphical representation. FIG. 3B is a representative plot depicting the association between baseline concentrations of SM 20:2 and longitudinal cognitive (ADAS-Cog13) and imaging (MRI: brain ventricular volume) changes during follow-up. Lines represent trajectories on subjects on the 25th percentile (black line), 50th percentile (red line), 75th percentile (green line) of baseline SM 20:2. Y-axes are ADAS-Cog13 score (left panel) and ventricular volume (right panel). Trajectories for these values are calculated based on the studied mixed-effects models. Abbreviations: AD, Alzheimer's disease; ADAS-Cog13, Alzheimer's Disease Assessment Scale-Cognition; MCI, mild cognitive impairment; MRI, magnetic resonance imaging.

FIGS. 4A-4B include representative network models showing metabolic pathways correlated with the temporal evolution of biomarkers and clinical variables in AD. FIG. 4A is a partial correlation network. Gaussian graphical model of metabolite concentrations showing reconstructed metabolic pathways and highlighting of the different modules involved in the steps along the temporal evolution of biomarkers and clinical variables in AD. Nodes in the network represent the metabolites, and edges (lines) illustrate the strength and direction of their partial correlations. Only partial correlations significant after Bonferroni correction for all possible edges are included. Labels show the major classes of metabolites included in our study. Gray circles outline the modules highlighted in panel B. FIG. 4B includes a representative schematic diagram of the model of temporal evolution of biomarkers in AD, augmented with colored versions of the network from FIG. 4A. In these networks, nodes are highlighted according to the strength and direction of the metabolite's association with the respective clinical trait with blue as positive and red as negative (networks in temporal order from left to right: pathological Aβ₁₋₄₂, T-tau, SPARE-AD, and ADAS-Cog13). Significant associations are colored in dark blue/bright red, and weaker (but at least nominally significant at 0.05) associations are displayed in fainter colors. Modules of metabolites implicated in the respective trait are highlighted by circles colored by their first occurrence in the temporal order following the color scheme of the time sequence on the bottom. The partial correlation network for Aβ₁₋₄₂ (FIG. 4A) highlighted direct correlations with short- and medium-chain SM and PC with ether bonds suggesting a role for membrane structure and function, contact sites, and membrane signaling in amyloid pathology. There was a different pattern for tau (FIG. 4B) with highlighted metabolites with long-chain acylcarnitines and SM implicated in lipid metabolism showing association with T-tau level. The SPARE-AD and ADAS-Cog13 partial correlation networks were very similar suggesting associations of brain atrophy and cognitive decline with metabolic changes in BCAAs and short-chain acylcarnitines that have been implicated in mitochondrial energetics as well as additional changes in lipid metabolism. Abbreviations: AD, Alzheimer's disease; ADAS-Cog13, Alzheimer's Disease Assessment Scale-Cognition; BCAA, branched-chain amino acid; PC, glycero-phosphatidylcholines (aa 5 diacyl, ae 5 acyl-alkyl); SM, sphingomyelin; SPARE-AD, Spatial Pattern of Abnormalities for Recognition of Early AD.

FIG. 5 is a representative diagram of a coexpression subnetwork with direct and indirect interconnections between select metabolites. The coexpression subnetwork focused on three metabolites also identified in the Rotterdam data set (PC ae C40:3, valine, and SM C20:2) was generated from a primary network (not shown). The subnetwork shows these three metabolites have high correlations (red edges lines) and lower correlations (green edges lines) to multiple modules via direct and indirect interconnections. Each module is denoted by a color representing a robust set of coregulated metabolites in interconnected biochemical pathways, for example, orange module contained a subset of amines, green module consists of long-chain acylcarnitines; teal, brown, and blue modules contained exclusively PC and lysoPC; red module contained SM and PC; gray module contained short-chain acylcarnitines and other amines. Each node represents a metabolite. The edge (line) opacity is proportional to the Pearson correlation, that is, lighter means weaker correlation value and darker means stronger correlation. The intermodule edges represent correlations and potentially indirect interactions among metabolites and biochemical pathways. The coexpression network captures all significant associations between metabolites and reveals a global correlation structure and interconnections among different modules that adds to our understanding of the disease network. Abbreviations: lysoPC, lyso-glycero-phosphatidylcholines (a 5 acyl); PC, glycero-phosphatidylcholines (aa 5 diacyl, ae 5 acyl-alkyl); PC ae, ether-containing PC; SM, sphingomyelin.

FIG. 6 is a representative flow chart of included and excluded subjects in the ANDI-1 cohort study.

FIG. 7 is representative co-expression network with direct and indirect interconnections between metabolites. Co-expression network showing the formation of 7 modules. Each module is denoted by a color representing a robust set of co-regulated metabolites in interconnected biochemical pathways (orange module contained a subset of amines, green module consists of long chain acylcarnitines, brown and blue modules contained exclusively PC and lyso PC, red module contained SM and PC, grey module contained short chain acylcarnitines and other amines). Each node represents a metabolite. The edge (line) opacity is proportional to the Pearson correlation (lighter means weaker correlation value and darker means stronger correlation). The inter-module edges represent correlations and potentially indirect interactions among metabolites and biochemical pathways. The co-expression network captured all significant associations between metabolites and revealed a global correlation structure and interconnections among different modules that can add to our understanding of disease network failures. Notably, many PC correlated with SM C16:0. There were many indirect interactions between amines, short/long-chain acylcarnitines, PC, and SM suggesting that related metabolic failures might underlie the associations we observed with cognitive and biomarker changes. Valine was correlated with α-AAA and isoleucine, which in turn connected with a short-chain acylcarnitines (C3). C3 connected with other short-chain acylcarnitines to form a fully connected clique. Then, C2 correlated with long-chain acylcarnitines which, in turn, connected with SM and PC.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, the terms “subject” and “patient” are used interchangeably irrespective of whether the subject has or is currently undergoing any form of treatment. As used herein, the terms “subject” and “subjects” refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous monkey, chimpanzee, etc.) and a human). In some aspects, the subject is a human.

The terms “treat,” “treated,” or “treating,” as used herein, refer to a therapeutic method wherein the object is to slow down (lessen) an undesired physiological condition, disorder or disease, or to obtain beneficial or desired clinical results. In some aspects of the present disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment also includes prolonging survival as compared to expected survival if not receiving treatment.

Before any embodiments of the present disclosure are explained in detail, it is to be understood that the present disclosure is not limited in its application to the details of construction and the arrangement of components set forth in the following description or illustrated in the accompanying drawings. The present disclosure is capable of other embodiments and of being practiced or of being carried out in various ways.

Other aspects of the invention will become apparent by consideration of the detailed description and accompanying drawings.

Embodiments of the present disclosure relate generally to the analysis and identification of global metabolic changes in Alzheimer's disease (AD). More particularly, the present disclosure provides materials and methods relating to the use of metabolomics as a biochemical approach to identify peripheral metabolic changes in AD patients and correlate them to cerebrospinal fluid pathology markers, imaging features, and cognitive performance.

The present disclosure, baseline serum samples were profiled from the Alzheimer's Disease Neuroimaging Initiative-1 (ADNI-1) cohort where vast data exist on each patient including cognitive decline and imaging changes over many years, information on CSF markers, genetics, and other-omics data. CSF biomarkers were used to define early metabolic changes in cognitively normal participants who have CSF pathology and to evaluate metabolic signatures that might be related to Aβ₁₋₄₂ and tau pathology. Using partial correlation networks, progressive metabolic changes were defined that accompany changes in CSF Aβ₁₋₄₂, CSF tau, brain structure, and cognition, whereas coexpression networks were used to connect key metabolic changes implicated in disease. The relationship of metabolites with longitudinal cognitive and imaging changes helped us define metabolic signatures correlated with disease progression. Key associations were also present in multiple independent cohorts. The systems approach described in the present disclosure facilitated the elucidation of metabolic changes along different stages during the progression of AD, and led to the identification of valuable peripheral biomarkers that can inform and accelerate clinical trials.

The present disclosure provides the biochemical knowledge about disease mechanisms that can be used as a roadmap for novel drug discovery and establishment of blood-based biomarkers. Eight complementary, targeted and non-targeted, metabolomics platforms are currently in the process of generating data on ADNI participants to define the metabolic trajectory of disease connecting central and peripheral metabolic failures in a pathway and network context. The present disclosure expands on biochemical coverage to better understand disease pathogenesis by using complementary data unique to ADNI-1. The unique opportunity of having longitudinal cognitive and imaging data on each subject for close to a decade enables identification of peripheral biomarkers that are disease related.

Accordingly, the present disclosure represents the first use of a targeted, highly validated metabolomics platform with the analysis guided by CSF markers and imaging data. Using 732 base-line serum samples from the ADNI-1 cohort, relationships between metabolomics data and cross-sectional clinical, CSF, and MRI measures were systematically evaluated, as well as their association with longitudinal cognitive and brain volume changes. Multiple comparisons and covariate-adjusted analyses, that included relevant medications, identified sets of metabolites that became altered at specific disease stages (preclinical AD with biomarker-defined AD pathology vs. symptomatic stages). Using partial correlation networks, the results of the present disclosure integrates data on the metabolic effects on AD pathogenesis, linking central and peripheral metabolism in a way that consistently addresses biochemical trajectories of disease with this established temporal sequence of pathophysiological stages of AD.

Aβ Pathology. Embodiments of the present disclosure identified changes in biomarker metabolites in early AD subjects, including biomarkers defined preclinical stages in CN participants, which were present in higher concentrations as compared to controls. These included a specific set of PCs (e.g., PC ae C36.2, PC ae C40.3, PC ae C42.4, and PC ae C44.4) and SMs (SM (OH) C14.1, SM C16.0). These biomarker metabolites were associated with abnormal CSF Aβ₁₋₄₂ values in CN subjects to a similar degree as observed in MCI subjects, indicating an early role of ether-containing PC species and SM in the development of Alzheimer's disease. In some cases, these metabolites were also associated with later cognitive decline and global brain atrophy changes in the MCI group (see, e.g., Table 1). The data of the present disclosure indicate imbalances and/or dysfunction with phospholipid metabolism in early phases of Alzheimer's disease progression. Partial correlation networks showed that the pathological CSF Aβ₁₋₄₂ values were associated with two groups of lipids, composed primarily of ether-containing PCs and relatively short-chain SMs. Ether-containing PC (PC ae) biomarker metabolites are PC species with an ether linkage of an aliphatic chain to the first hydroxyl position of glycerol. These lipids may represent a mixture of lipid metabolites including but not limited to, plasmalogens, acyl-alkyl PC, or PC containing an odd-numbered fatty acyl chain. When measured in a biological sample such as serum, for example, ether-containing lipids are derived from liver metabolism and are possible indicators of peroxisomal function and lipid oxidation status. Plasmalogens and SMs may be enriched in membrane rafts where they facilitate signal transduction and serve as a source for lipid secondary messengers. The association of PCs and SMs described in the present disclosure with early changes in AD and with pathological CSF Aβ₁₋₄₂ levels may be indicative of early neurodegeneration and loss of membrane function. Ether-linked PC biomarker metabolites may be found in high abundance in plasma membranes and are a source for signaling molecules, including platelet-activating factor and arachidonic acid. Similarly, they may be found in high abundance in immune cells, are regulatory factors, and may be part of a link between inflammation and AD. Both SMs and ether-linked PCs may be located in membrane rafts, suggesting that lipid rafts are directly associated with regulation of amyloid precursor protein processing, the production of Aβ₁₋₄₂, and facilitate its aggregation.

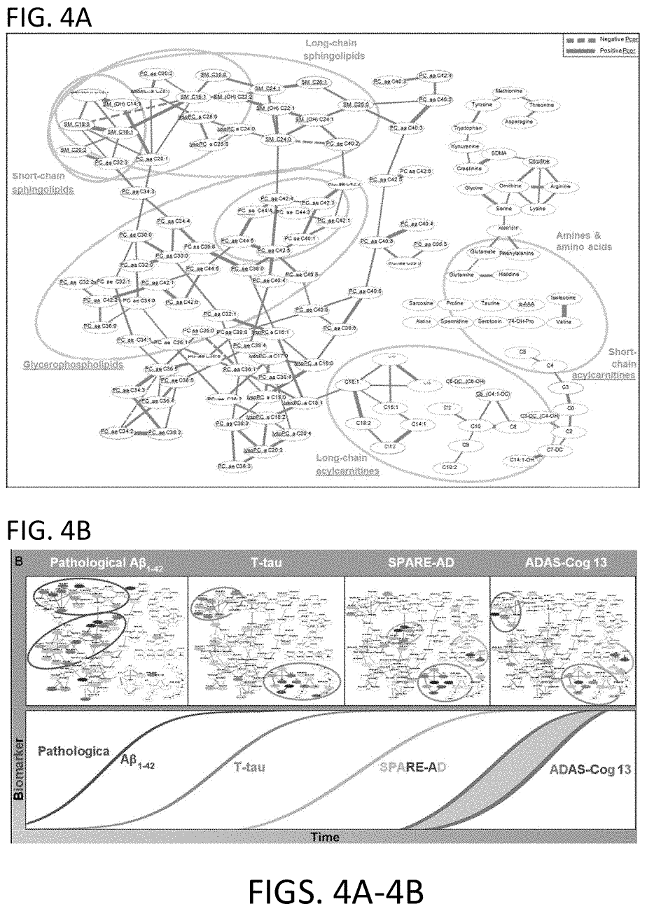

Tau pathology. In accordance with embodiments of the present disclosure, pathological CSF Aβ₁₋₄₂ shows an association with ether-linked PCs, and shorter chain SMs, but not amines, lysoPC, or acylcarnitines. Aβ₁₋₄₂ changes happen early in Alzheimer's disease, followed by accumulation of tau protein in the CSF. As described herein, tau-related biomarker metabolites were very different both from those that correlate with Aβ₁₋₄₂ as well as from metabolites associated with brain atrophy and cognitive changes. Tau-related metabolites may belong to an intermediate stage between Aβ₁₋₄₂ accumulation and changes in imaging and cognitive function (see, e.g., FIG. 4B), further demonstrating that different metabolic events occur at different disease stages. For example, as shown herein, long-chain acylcarnitines, PC ae C36:2, and SM.C20:2 were present in higher concentrations in cognitively impaired subjects, as compared to controls, with AD-like CSF Aβ₁₋₄₂ values, indicating that changes in these metabolites are more specific to AD-related neurodegeneration. Additionally, accumulation of acylcarnitine species containing long fatty acyl chains indicates malfunction of fatty acid transport and/or β-oxidation in mitochondria, inefficient utilization of fatty acids as energy substrates, and/or alterations in tau metabolism. As demonstrated in the present disclosure, levels of several acylcarnitine species were increased either at the MCI stage or in clinical AD (see, e.g., Table 1).

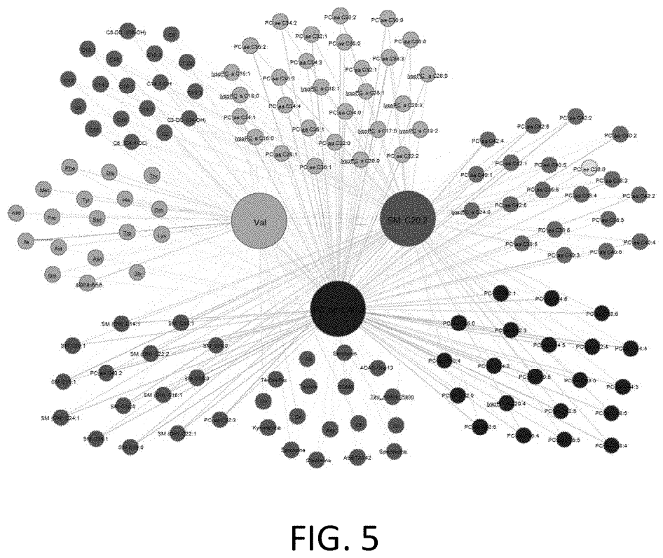

Brain volume changes and cognitive decline. In accordance with embodiments of the present disclosure, partial correlation networks can be used to show a pattern of inverse associations between brain volume changes (e.g., measured by SPARE-AD) and cognition (ADAS-Cog13), and long and short acylcarnitines, valine, and a-AAA, indicating a shift in energy substrate utilization in later stages of AD (see, e.g., FIG. 4). Using a coexpression network, data of the present disclosure shows a relationship between valine and short acylcarnitines (see, e.g., FIG. 5). The association of the long-chain acylcarnitines, odd-numbered acylcarnitines, and amino acids in relation with ADAS-Cog scores may indicate a switch of utilization from fatty acids to amino acids and glucose. In network analysis described herein, the amines and short-chain acylcarnitines did not link to PCs and SMs, rather they clustered together in smaller groups. This may indicate that the short-chain acylcarnitines are associated in energy and amino acid metabolism rather than lipid metabolism in AD subjects. This demonstrates a disease-associated transition in pathways for utilization of energy substrates.

The present disclosure provides the material and methods pertaining to the use of metabolomics and network approaches to identify lipid metabolic changes related to early stages of AD, as well as later changes related to mitochondrial energetics and energy utilization. The lipid changes identified herein reflect alterations in membrane structure and function early in the disease process and suggest a change in lipid rafts, which in turn, cause alterations in AR processing. Over time, the changes in lipid membranes, particularly mitochondrial membranes, may result in increased lipid oxidation, loss of membrane potential, and changes in membrane transport. In some cases, lipid membrane changes might involve disruptions in BCAA as an energy source, production of acylcarnitines, and altered energy substrate utilization.

Amino acids are the monomeric building blocks of proteins, which in turn comprise a wide range of biological compounds, including enzymes, antibodies, hormones, transport molecules for ions and small molecules, collagen, and muscle tissues. Amino acids are considered hydrophobic or hydrophilic, based upon their solubility in water, and, more particularly, on the polarities of their side chains. Amino acids having polar side chains are hydrophilic, while amino acids having nonpolar side chains are hydrophobic. The solubilities of amino acids, impart, determines the structures of proteins. Hydrophilic amino acids tend to make up the surfaces of proteins while hydrophobic amino acids tend to make up the water-insoluble interior portions of proteins. Of the common 20 amino acids, nine are considered essential in humans, as the body cannot synthesize them. Rather, these nine amino acids are obtained through an individual's diet. A deficiency of one or more amino acids can cause various imbalances and can lead to the development of a disease condition(s). Additionally, as described herein, the presence or absence of one or more amino acids can indicate metabolic imbalances reflective of disease conditions, such as Alzheimer's disease. Branched chain amino acids (BCAAs), which include valine, leucine, and isoleucine, are among a subgroup of amino acids that can be predictive of the development of Alzheimer's disease. As such, BCAAs can be used to treat such conditions as they have been shown to function not only as protein building blocks, but also as inducers of signal transduction pathways that modulate translation initiation.

In some cases, several ether-linked PC metabolites have been associated with a risk of diabetes; insulin resistance may promote aminoacidemia and the use of amino acids for energy, and BCAA and a-AAA have been identified as predictors of diabetes risk. BCAAs (e.g., valine, leucine, and isoleucine) are important for balanced metabolism and have been implicated in insulin resistance, type-2 diabetes mellitus, and obesity. As described herein, low levels of valine and its correlation with cognitive changes were demonstrated, pointing to an important role for this BCAA in cognitive changes in AD. Low levels of BCAAs have been implicated in hepatic insulin resistance in liver disease and may have a broader role in insulin resistance in the brain.

In some embodiments of the present disclosure, it may be desirable to include a control sample. The control sample may be analyzed concurrently with the sample from the subject as described above. The results obtained from the subject sample can be compared to the results obtained from the control sample. Standard curves may be provided, with which assay results for the sample may be compared. Such standard curves present levels of biomarker as a function of assay units (e.g., fluorescent signal intensity, biochemical indicator). Using samples taken from multiple donors, standard curves can be provided for reference levels of a biomarker metabolite in subjects with normal cognition, for example, as well as for “at-risk” levels of the biomarker metabolite (e.g., MCI subjects) in samples obtained from donors, who may have one or more of the characteristics set forth above.

In accordance with these embodiments, a method for determining the presence, amount, or concentration of a biomarker metabolite in a test sample is provided. The method comprises assaying a test sample and/or a control sample for a biomarker metabolite using an assay, for example, designed to detect the metabolite itself (e.g., detectable label) and/or using an assay that compares a signal generated by a detectable label as a direct or indirect indication of the presence, amount, or concentration of a biomarker metabolite in the test sample to a signal generated as a direct or indirect indication of the presence, amount, or concentration of a control.

In some embodiments, the present disclosure provides a method for diagnosing or detecting Alzheimer's disease in a subject. In accordance with these embodiments, the method includes obtaining a sample from a subject (e.g., serum sample) and performing biochemical analysis on the sample to detect the presence of at least one biomarker metabolite. In some cases, the biomarker metabolite is a carnitine biomarker metabolite, a phosphatidylcholine biomarker metabolite, a sphingomyelin biomarker metabolite, or any combinations thereof. Detecting the biomarker metabolite can then be used to establish an association with the subject having at least one independent indicator of Alzheimer's disease, such that the subject is diagnosed with having Alzheimer's disease if at least one biomarker metabolite is detected. The method may also include administering a treatment to alleviate one or more symptoms of AD, and may also include assessing the biomarker metabolite again in order to determine if the treatment is therapeutically beneficial.

In some embodiments, the present disclosure provides methods for diagnosing or detecting Mild Cognitive Impairment (MCI) in a subject, and/or distinguishing between early phases of AD from late states of AD. In accordance with these embodiments, the method includes obtaining a sample from a subject (e.g., serum sample) and performing biochemical analysis on the sample to detect the presence of at least one biomarker metabolite. In some cases, the biomarker metabolite is a carnitine biomarker metabolite, a phosphatidylcholine biomarker metabolite, a sphingomyelin biomarker metabolite, or combinations thereof. Detecting the biomarker metabolite can then be used to establish an association with the subject having at least one independent indicator of MCI. The method may also include administering a treatment to alleviate one or more symptoms of MCI, and may also include assessing the biomarker metabolite again in order to determine if the treatment is therapeutically beneficial.

In still other embodiments, the present disclosure provides a method for predicting the outcome of a subject suspected having AD. In accordance with these embodiments, the method includes obtaining a sample from a subject and performing biochemical analysis on the sample to detect the presence of at least one biomarker metabolite. In some cases, the biomarker metabolite is a carnitine biomarker metabolite, a phosphatidylcholine biomarker metabolite, a sphingomyelin biomarker metabolite, or combinations thereof. The method may also include assessing at least one independent indicator of AD in the subject, such that detection of the at least one biomarker metabolite is associated with the subject having at least one independent indicator of AD. In some cases, the subject is predicted to develop AD if at least one biomarker metabolite is detected. The method may also include administering a treatment to alleviate one or more symptoms of MCI, and may also include assessing the biomarker metabolite again in order to determine if the treatment is therapeutically beneficial.

In some embodiments, the absolute amount of a biomarker metabolite is correlated with subjects having varying degrees of AD progression (e.g., from normal cognition to MCI). In some embodiments, the absolute amount of a biomarker metabolite is correlated with an assessment score such as an Alzheimer's Disease Assessment Scale cognitive subscale 13 (ADAS-Cog 13) score, or a Spatial Pattern of Abnormality for Recognition of Early Alzheimer's disease (SPARE-AD) score. In some embodiments, the absolute amount of a biomarker metabolite is correlated with subjects having MCI.

As described and used herein, “sample,” “test sample,” and “biological sample” refer to fluid sample containing or suspected of containing a biomarker metabolite. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing a biomarker metabolite may be assayed directly. In one embodiment, the source containing a biomarker metabolite is a human bodily substance (e.g., bodily fluid, blood such as whole blood, serum, plasma, urine, saliva, sweat, sputum, semen, mucus, lacrimal fluid, lymph fluid, amniotic fluid, interstitial fluid, lung lavage, cerebrospinal fluid, feces, tissue, organ, or the like). Tissues may include, but are not limited to skeletal muscle tissue, liver tissue, lung tissue, kidney tissue, myocardial tissue, brain tissue, bone marrow, cervix tissue, skin, etc. The sample may be a liquid sample or a liquid extract of a solid sample. In certain cases, the source of the sample may be an organ or tissue, such as a biopsy sample, which may be solubilized by tissue disintegration/cell lysis.

### EXAMPLES

Metabolomic analyses were performed in the ADNI-1 cohort, and key findings were further tested in the Rotterdam, EFR, and IMAS cohorts. Overall descriptions of sample size, composition, and studied outcomes across the different cohorts are shown in Table 6. The results are presented for each cohort in the following Examples.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties. The present disclosure has multiple aspects, illustrated by the following non-limiting examples.

### Example 1: ADNI-1 Cohort

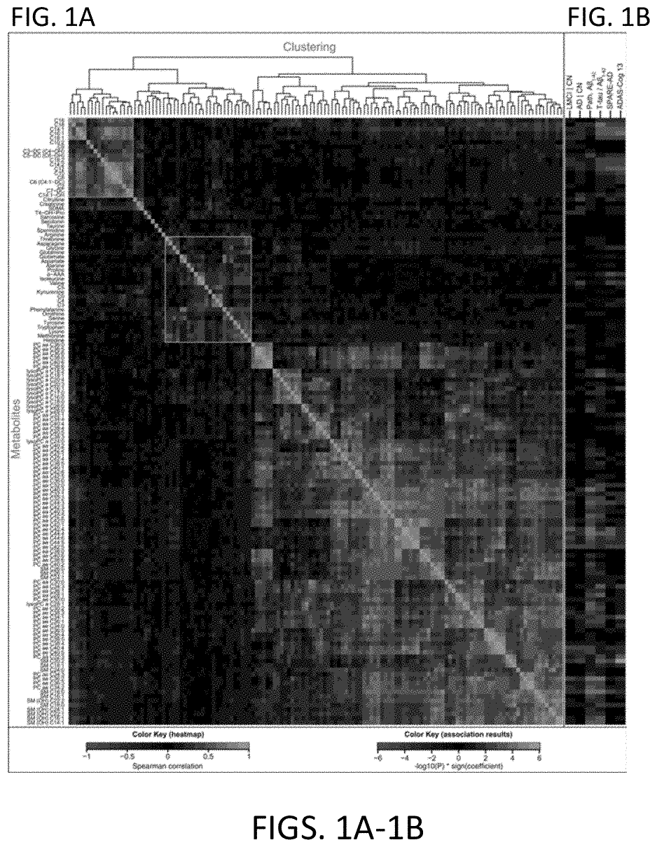

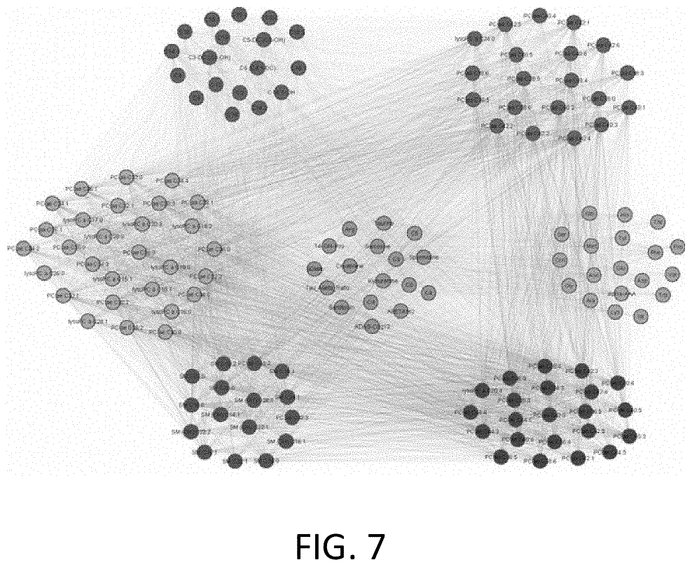

In ADNI-1, CN, MCI, and AD subjects did not differ in mean age but, as expected, differed in APOE ε4 frequency, baseline cognition, MRI atrophy index, and CSF levels of T-tau and Aβ₁₋₄₂. The representative heat map shown in FIG. 1 shows that the global (direct and indirect) correlation structure between biomarker metabolites can be formed into biochemical classes, illustrating that the biomarker metabolites with significant findings can be seen as proxies for the group of their correlating metabolites (see also FIG. 7).

### Example 2: ADNI-1: Metabolites Associated with Cross-Sectional Clinical, MRI, and CSF Biomarker Measures

The biomarker metabolites that remained in the analyses after the QC steps showed different correlation strengths, indicating groups of metabolites that may be involved in similar processes (FIG. 1). After applying Bonferroni multiple comparison correction, 13 metabolites showed significant associations (Bonferroni-adjusted P-value<05) with cognitive scores and CSF and MRI biomarker measures (Table 1). Six metabolites were associated with CSF Aβ₁₋₄₂ positivity (ether-containing PC [PC ae] C36:2, PC ae C40:3, PC ae C42:4, PC ae C44:4, SM (OH) C14:1, SM C16:0), four were associated with t-tau/Aβ₁₋₄₂ ratio (C18, PC ae C36:2, SM C16:0, SM C20:2), five were associated with ADAS-Cog13 scores (C14:1, C16:1, SM C20:2, α-aminoadipic acid [α-AAA], and valine), and 6 were associated with SPARE-AD scores (C12, C16:1, PC ae C42:4, PC ae C44:4, α-AAA, and valine). In all analyses, higher acylcarnitine, PC, and sphingomyelin (SM) values were associated with worse clinical and biomarker measures, whereas the opposite direction of associations was observed for valine and α-AAA values. The complete results for the 138 studied metabolites are listed in Table 10, where many amines (including isoleucine, glutamate, tyrosine, tryptophan, glycine, proline, histidine, T4OH proline) and other metabolites within PC and SM classes showed significant non-comparison-corrected associations with clinical markers and outcomes but did not survive Bonferroni multiple comparison correction. All significant correlations were in the same directions in the clinical diagnostic groups (Tables 11 and 12).

TABLE 1
Metabolites associated with clinical diagnosis, MRI, or CSF biomarkers after Bonferroni correction.

| Metabolites | MCI | | AD | | Aβ1-42 | | T-tau/Aβ1-42 | | ADAS-Cog13 | | SPARE-AD | |
|---|---|---|---|---|---|---|---|---|---|---|---|---|
| C12 | 0.9 | (1.0) | −1.62 | (1.0) | 1.22 | (1.0) | 0.26 | (.33) | 5.88 | (.073) | 0.87 | (.041) |
| C14:1 | 10.79 | (1.0) | −12.25 | (1.0) | 12.93 | (1.0) | 2.46 | (.05) | 52.21 | (.037) | 6.8 | (.1) |
| C16:1 | 1.25 | (1.0) | −22.098 | (1.0) | 1.62 | (1.0) | 0.38 | (.091) | 9.4 | (.0037) | 1.2 | (.020) |
| C18 | 14.62 | (1.0) | −19.27 | (1.0) | 21.62 | (1.0) | 4.64 | (.0055) | 64.31 | (.5) | 10.0095 | (.2) |
| PC ae C36:2 | 0.085 | (.33) | −0.082 | (1.0) | 0.16 | (.007) | 0.018 | (.013) | 0.23 | (1.0) | 0.027 | (1.0) |
| PC ae C40:3 | 0.98 | (1.0) | −3.27 | (1.0) | 5.76 | (.017) | 0.49 | (.55) | 2.72 | (1.0) | 0.26 | (1.0) |
| PC ae C42:4 | 1.62 | (.063) | −1.51 | (.88) | 2.32 | (.017) | 0.19 | (.75) | 3.63 | (1.0) | 0.79 | (.049) |
| PC ae C44:4 | 3.029 | (1.0) | −3.37 | (1.0) | 6.11 | (.016) | 0.6 | (.089) | 11.24 | (.64) | 2.059 | (.037) |
| SM (OH) C14:1 | 0.06 | (1.0) | −0.054 | (1.0) | 0.24 | (.044) | 0.027 | (.081) | 0.2 | (1.0) | 0.016 | (1.0) |
| SM C16:0 | 0.0065 | (1.0) | −0.0074 | (1.0) | 0.015 | (.016) | 0.0017 | (.013) | 0.024 | (1.0) | 0.0037 | (.57) |
| SM C20:2 | 0.66 | (1.0) | −1.082 | (.22) | 0.74 | (1.0) | 0.18 | (.047) | 4.57 | (<0.0001) | 0.4 | (.48) |
| α-AAA | −0.46 | (1.0) | 0.67 | (1.0) | −0.68 | (1.0) | −0.13 | (.098) | 3.7 | (0.0025) | −0.61 | (<0.0001) |
| Valine | −0.0038 | (1.0) | 0.0073 | (.079) | −0.004 | (1.0) | −0.0006 | (1.0) | −0.028 | (<0.0001) | −0.0039 | (<0.0001) |

Abbreviations: MRI, magnetic resonance imaging; CSF, cerebrospinal fluid; MCI, mild cognitive impairment; AD, Alzheimer's disease; ADAS-Cog13, Alzheimer's Disease Assessment Scale-Cognition; SPARE-AD, Spatial Pattern of Abnormalities for Recognition of Early AD; α-AAA, α-aminoadipic acid.
NOTE.
The cells include the logistic (MCI and AD) and linear (Aβ₁₋₄₂, T-tau/Aβ₁₋₄₂, ADAS-Cog13, SPARE-AD) regression coefficients and, in parenthesis, the Bonferroni corrected P-value. All model included age and gender as covariates. APOE ε4 presence included in Aβ₁₋₄₂ model and education was included in the MCI, AD, and ADAS-Cog13 models.

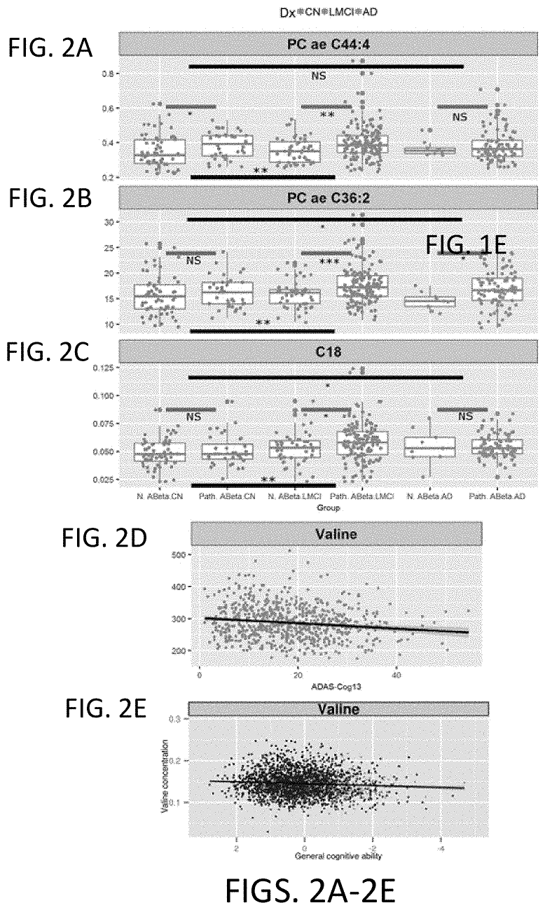

In several embodiments, differences in levels of key metabolites associated with cognitive or biomarker measures were evaluated from the analyses reported previously between the three diagnostic groups (CN, MCI, and AD) subclassified by CSF Aβ₁₋₄₂ positivity status. Metabolites showed three different patterns of associations with the CSF AD biomarkers. PC ae C44:4, PC ae C36:2, and C18 represented the most significant examples of each of these patterns, and the values in the six groups are shown in FIG. 2. In some cases, CN subjects (red boxes) with pathological CSF Aβ₁₋₄₂ values showed significant metabolic changes in a specific group of metabolites compared with CN with no pathological CSF Aβ₁₋₄₂ values (FIG. 2A). Some of the changes associated with CSF Aβ₁₋₄₂ values appeared in clinical stages of disease (MCI and AD; FIG. 2B). Other metabolic changes were only observed in comparing CN participants to clinically impaired subjects (FIG. 2C) but showed no associations with pathological CSF Aβ₁₋₄₂ status. FIG. 2D illustrates valine correlation with cognition in the ADNI-1 study.

### Example 3: Metabolites Associated with Longitudinal Outcomes in the ADNI-1 Cohort

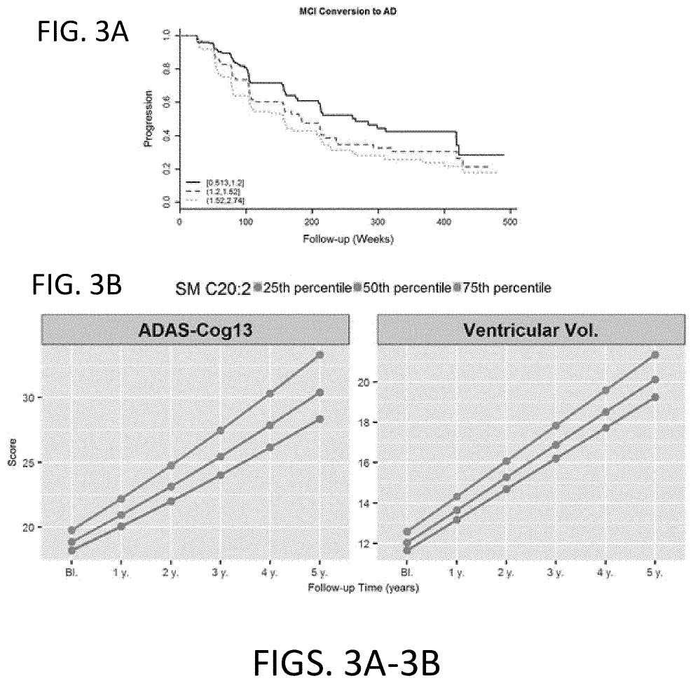

Levels of metabolites at baseline were evaluated for association with (1) ADAS-Cog13 changes up to 5 years; (2) ventricular volume changes up to 5 years; or (3) progression from MCI to AD (Table 2). Regression coefficients of six metabolites (PC ae C40:3, PC ae C42:4, PC ae C44:4, SM (OH) C14:1, SM C16:0, and SM C20:2) showed a positive association with all three longitudinal outcomes. In addition, lower valine and α-AAA values were associated with faster cognitive decline; similarly, the coefficient for valine was negatively associated with ventricular volume changes. FIG. 3 shows some of these associations as examples, including FIG. 3A which shows the Cox hazards model of the association of SM C20:2 with conversion from MCI to AD, and FIG. 3B which shows the association between baseline concentration of SM 20:2 (presented as tertiles) and longitudinal cognitive (ADAS-Cog13) and MRI (brain ventricular volume) change.

TABLE 2
Association of metabolites with longitudinal cognitive and MRI changes in MCI.

| | | | | | Progression MCI to AD | |
|---|---|---|---|---|---|---|
| Metabolites | ADAS-Cog13 Change | | Ventricle Volume Change | | Dementia | |
| C12 | 0.091 | (0.26) | 0.11 | (0.73) | 1.37 | (0.4) |
| C14:1 | 1.39 | (0.034) | 7.085 | (0.006) | 2.11 | (0.22) |
| C16:1 | 0.15 | (0.13) | 0.67 | (0.092) | 1.9 | (0.19) |
| C18 | −0.16 | (0.87) | 1.94 | (0.64) | 2.41 | (0.18) |
| PC ae C36:2 | 0.0075 | (0.094) | 0.031 | (0.096) | 1.056 | (0.012) |
| PC ae C40:3 | 0.38 | (0.02) | 1.5 | (0.020) | 5.98 | (0.027) |
| PC ae C42:4 | 0.15 | (0.04) | 0.72 | (0.013) | 1.96 | (0.042) |
| PC ae C44:4 | 0.49 | (0.0076) | 2.33 | (0.0012) | 5.98 | (0.027) |
| SM (OH) C14:1 | 0.015 | (0.04) | 0.075 | (0.01) | 1.08 | (0.025) |
| SM C16:0 | 0.0009 | (0.025) | 0.0037 | (0.023) | 1.004 | (0.029) |
| SM C20:2 | 0.11 | (0.0078) | 0.48 | (0.0035) | 1.9 | (0.0023) |
| α-AAA | −0.093 | (0.022) | −0.29 | (0.087) | 0.68 | (0.061) |
| Valine | −0.0006 | (0.035) | 0.0027 | (0.026) | 1.0 | (0.27) |

Abbreviations: MRI, magnetic resonance imaging; MCI, mild cognitive impairment; ADAS-Cog13, Alzheimer's Disease Assessment Scale-Cognition; AD, Alzheimer's disease; PC ae, ether-containing PC; α-AAA, α-aminoadipic acid. Table depicts the association between selected metabolites and longitudinal ADAS-Cog13 (column 2) and ventricular volume (column 3) in mixed-effects models that were age, gender, and APOE adjusted. In addition, the ADAS-Cog13 model was adjusted for education. Boxes contain the coefficients and, in parenthesis, the P-values. The last column (column 4) presents the associations of the metabolites with progression from MCI to AD in Cox hazards models that included age, gender, education, and APOE as covariates. Values represent hazard
ratio and, in parenthesis, the P-values. Significant associations are bolded for an easier visualization. All P-values were not multiple comparison-corrected.

### Example 4: Evaluation of Results in the Rotterdam and ERF Studies

In the Rotterdam and ERF studies, only a subset of metabolites was measured from the panel of P180 metabolites evaluated in the ADNI-1 study (P150 panel; Table 13). Using a targeted approach, the metabolites that showed a significant association in the ADNI-1 study were tested and were also correlated with cognition (general cognitive ability: g-factor) in the Rotterdam Study or ERF. For the cross-sectional analysis, eight metabolites were available in the ERF study. Two of these metabolites (PC ae C40:3 and SM C20:2) were associated with cross-sectional general cognitive ability in the expected direction based on the discovery ADNI-1 cohort. Higher general cognitive ability levels indicate better cognition as opposed to ADAS-Cog13. Valine was strongly associated with a higher general cognitive ability (P=0.00035) in the Rotterdam study (FIG. 2E), which is in line with the association with ADAS-Cog13 in ADNI-1 (FIG. 2D). Longitudinally, 342 participants developed AD in the Rotterdam study after a median follow-up time of 9.7 years (IQR 5.6-10.5). A Cox proportional hazard model was fitted adjusting for age at baseline, gender, education, and lipid-lowering medication and indicated that a 1-SD increase in valine concentration was also associated with a decreased risk of AD (P=0.044).

### Example 5: Evaluation of Aβ₁₋₄₂ Signature in the IMAS Cohort

Three of the six metabolites (PC ae 42:4, PC ae 44:4, and SM (OH) C14:1) that showed an association with CSF Aβ₁₋₄₂ positivity in the ADNI-1 cohort were also associated with amyloid positivity on PET in the IMAS cohort (n=34; Table 14).

### Example 6: Partial Correlation Networks for Aβ₁₋₄₂, T-Tau, SPARE-AD, ADAS-Cog13-Metabolic Trajectory for Disease

FIG. 4 integrates the strength of the partial correlations between metabolites and overlays on these networks the associations with the studied outcomes Aβ₁₋₄₂, t-tau, SPARE-AD, and ADAS-Cog13 (partial correlation networks for p-tau and t-tau/Aβ₁₋₄₂ ratio are not shown). The networks showing the direct links between metabolites (nodes) identified through their strong partial correlations (edges) expand the heat map information association to CSF, imaging, and cognitive markers, respectively (where bright colors indicate strong associations and blue and red color indicate upregulation and downregulation of metabolites), these networks demonstrate how the effects of clinical variables propagate along the edges within the network suggesting that the results follow biochemically plausible pathways. The network for Aβ₁₋₄₂ (FIG. 4A) highlighted direct correlations with short- and medium-chain SMs and PC with ether bonds, suggesting a role for membrane structure and function, contact sites, and membrane signaling in amyloid pathology. The correlation pattern for t-tau (FIG. 4B) highlighted metabolites among long-chain acylcarnitines and SMs implicated in lipid metabolism. The SPARE-AD and ADAS-Cog13 (FIG. 4B) partial correlation networks were very similar, suggesting associations of brain atrophy and cognitive decline with metabolic changes in BCAAs and short-chain acylcarnitines implicated in mitochondrial energetics as well as additional changes in lipid metabolism.

### Example 7: Coexpression Network—Direct and Indirect Connections for Key Metabolites

The partial correlation networks evaluated direct connections among metabolites. To capture both indirect and direct correlations, built coexpression networks were generated to evaluate the number of modules in our data set and evaluate additional connections between key metabolites identified as related to cognitive or biomarker measures in ADNI-1. The correlation structure of the three metabolites was investigated in the ERF and Rotterdam data sets that significantly associated with cognition, namely PC ae C40:3, SM C20:2, valine as shown in FIG. 5. The subnetwork shows these three metabolites to have high correlations (marked as red edges) to other functional metabolic modules via direct and indirect links. Valine highly correlated with isoleucine and α-AAA, whereas SM C20:2 highly correlated with a subset of the SMs including SM C16:0. Finally, PC ae C40:3 highly correlated with PCs and SMs, but not amines and acylcarnitines. These SMs and PCs were significantly associated with cognitive scores, CSF biomarkers, and MRI measures (Table 1).

### Materials and Methods

ADNI-1 baseline samples. ADNI shipped 831 samples with unique identifiers belonging to 807 subjects. These initial identifiers were different from the ADNI subject identifiers. There were duplicate aliquots from the same CSF draw for 24 subjects to evaluate analytical performance. Only after the final raw data were submitted to ADNI, the information was obtained to link the samples identifier to the subject RID and identify the duplicates. Data were obtained from the ADNI database in September 2015 (adni.loni.usc.edu). ADNI-1 was launched in 2004 by the National Institute on Aging (NIA), the National Institute of Biomedical Imaging and Bioengineering, the Food and Drug Administration, private pharmaceutical companies, and nonprofit organizations. ADNI-1 patients underwent extensive clinical and cognitive testing, including the Alzheimer's Disease Assessment Scale-Cognition (ADAS-Cog13), which was used as a measure of general cognition in this analysis. AD dementia diagnosis was established based on the NINDS-ADRDA criteria for probable AD. Mild cognitive impairment (MCI) participants did not meet these AD criteria and had largely intact functional performance, meeting predetermined criteria for amnestic MCI. Controls were cognitively normal (CN) (Table 3). Additional details of participant selection criteria and protocol are available at adni-info.org. The study was approved by institutional review boards of all participating institutions, and written informed consent was obtained from all participants and/or authorized representatives before study commencement.

TABLE 3
Baseline demographics, clinical and biomarker data of ADNI subjects.

| | CN (n = 199) | | MCI (n = 358) | | AD (n = 175) | | p-value |
|---|---|---|---|---|---|---|---|
| Age (years) | 75.3 | (72.2-78.3) | 75.1 | (70.1-80.4) | 75.6 | (70.8-80.2) | 0.56 |
| Gender (% Male) | 49.7% | | 35.4% | | 48.6% | | 0.0008 |
| APOE ε4 (%) | 27.6% | | 53.1% | | 65.7% | | <0.0001 |
| MMSE | 29.0 | (29.0-30.0) | 27.0 | (25.8-28.0) | 23.0 | (22.0-25.0) | <0.0001 |
| ADAS-Cog13 | 9.33 | (5.7-12.3) | 18.3 | (14.7-23.0) | 28.0 | (23.3-34.0) | <0.0001 |
| SPARE-AD | −1.36 | [(−1.87)-(−0.91)] | 0.67 | (0.05-1.38) | 1.35 | (0.82-1.74) | <0.0001 |
| Aβ₁₋₄₂ | 217.0 | (159.8-256.5) | 146.0 | (125.8-190.0) | 137.5 | (121.8-160.5) | <0.0001 |
| T-Tau | 62.0 | (49.5-86.0) | 86.0 | (65.0-123.0) | 111.0 | (79.0-152.0) | <0.0001 |
| P-Tau₁₈₁ | 21.0 | (16.0-30.0) | 31.0 | (21.0-45.5) | 36.0 | (29.0-49.3) | <0.0001 |

AD: Alzheimer disease; CN: cognitively normal; MCI: mild cognitive impairment; MMSE: mini mental state examination; SPARE-AD: Spatial Pattern of Abnormalities for Recognition of Early AD.

ADNI cohort. The primary goal of ADNI has been to test whether serial MRI, PET, other biological markers, and clinical and neuropsychological assessment can be combined to measure the progression of MCI and early AD. Determination of sensitive and specific markers of very early AD progression is intended to aid researchers and clinicians to develop new treatments and monitor their effectiveness, as well as lessen the time and cost of clinical trials. ADNI is the result of efforts of many co-investigators from a broad range of academic institutions and private corporations, and subjects have been recruited from over 50 sites across the U.S. and Canada. The initial goal of ADNI was to recruit 800 subjects but ADNI has been followed by ADNI-GO and ADNI-2. To date these three protocols have recruited over 1500 adults, ages 55 to 90, to participate in the research, consisting of cognitively normal older individuals, people with early or late MCI, and people with early AD. The follow up duration of each group is specified in the protocols for ADNI-1, ADNI-2 and ADNI-GO. Subjects originally recruited for ADNI-1 and ADNI-GO had the option to be followed in ADNI-2.

IMAS Cohort. Basic demographics, medication/medical history and genetic data were also available for all participants. At each visit, a detailed neuropsychological test battery was administered, serum samples before breakfast after overnight fasting were collected, and structural and functional MRI data were obtained; [¹¹C]PiB positron emission tomography (PET) for quantitation of amyloid beta plaque burden was also available for a subset of participants.

Rotterdam and Erasmus Rucphen Family cohorts. Participants from the Erasmus Rucphen Family (ERF) study (N5905) were metabolically profiled from fasting blood samples using the Biocrates AbsoluteIDQ-p150 kit platform, which measures a subset of metabolites from the P180 and excludes many of the amines. A previously described quality control (QC) protocol was applied. Valine was measured in fasting blood samples using the Brainshake platform in 2752 participants from the Rotterdam large prospective cohort study. Participants of the ERF study underwent a standardized cognitive test battery at the study center on the same day blood was drawn. Participants of the Rotterdam study underwent cognitive tests at the time of valine measurement, and all participants were followed up for AD clinical diagnosis.

TABLE 4
Characteristics of Rotterdam and ERF study.

| | Rotterdam study | | ERF study | |
|---|---|---|---|---|
| N-subjects | 2752* | | 905 | |
| Age (years) | 74.2 | (6.2) | 48 | (14.2) |
| Women (%) | 58.2 | | 56.3 | |
| Education (1-4 scale) | 2.4 | (0.9) | 2.1 | (0.9) |
| BMI (kg/m2) | 27.4 | (4.1) | 26.9 | (4.8) |

| | | |
|---|---|---|
| Lipid lowering Medications (%) | 22.8 | 11.7 |
| APOE ε4 carriers (%) | 27.6 | 36.9 |

*of which 2505 individuals had general cognitive ability measured

Rotterdam study is a prospective ongoing population based elderly cohort that started in 1990 in Ommoord, a district of Rotterdam. Participants are re-invited to undergo home interviews, fasted blood sampling and cognitive examinations at the research center every 4 years. Research presented is based on the participants in the fourth visit from the baseline cohort. The Rotterdam Study has been approved by the Medical Ethics Committee of the Erasmus MC and by the Ministry of Health, Welfare and Sport of the Netherlands, implementing the Wet Bevolkingsonderzoek: ERGO (Population Studies Act: Rotterdam Study). All participants provided written informed consent to participate in the study and to obtain information from their treating physicians. General cognitive ability was calculated as the first unrotated principal component of five cognitive tests in the Rotterdam study. The Stroop 3 (time needed to complete Stroop color-word card), letter digit substitution test, phonemic fluency tests, 15-word Auditory Verbal Learning Test (delayed recall) and the pegboard test (sum of left hand, right hand and both hands). Tests were coded such that a higher score of general cognitive ability depicts a better cognitive function. Participants of the Rotterdam Study were continuous followed-up through screening of general practitioner records and cognitive screening every 3-4 years at the research center. In the Rotterdam study the dementia status was assessed at each visit and death of subjects were continuously reported through automatic linkage with general practitioner files.

Valine was measured in the Rotterdam study by an NMR-based metabolomics analyses performed with the comprehensive quantitative serum/plasma platform described originally by Soininen et al. 2009; 2015. Valine was associated to general cognitive ability adjusting for age, sex, education attainment and lipid lowering medication in 2505 individuals. Valine was also associated in 2752 individuals with incident Alzheimer's disease in a Cox proportional hazards model adjusting for age at baseline, sex, education attainment and lipid lowering medication.

Participants visited the ERF study center in the period 2002-2006 and underwent in one day extensive testing on traits related to common complex diseases, including a cognitive test battery recorded by trained personnel. Participants not fasting at blood draw were excluded from the analysis. The ERF study was approved by the Medical Ethics Committee of the Erasmus MC. The committee is constituted according to the WMO (National act medical-scientific research in human beings). A written informed consent was obtained from all study participants. Targeted metabolomic measurements were performed using electrospray—flow injection analysis—tandem mass spectrometry methods and the Biocrates AbsoluteIDQ p150 kit (BIOCRATES Life Sciences AG). Quality control is described in detail elsewhere. General cognitive ability was calculated from the following tests; Stroop 3 (time needed to complete Stroop color-word card), 15-word Auditory Verbal Learning Test (sum of immediate (5 iterations) and delayed recall (once)), phonemic fluency (with D,A,T, number of words mentioned beginning with each letter, one minute each, sum of the three trials), TMT-B (time needed to complete Trail-making Test part B) and the WAIS block design test (number of correct answers, Wechsler scoring). In total, 905 subjects were available for analysis with general cognitive ability for this study.

The general cognitive ability or “g-factor” was calculated using previously described methods in dementia-free participants with available cognitive tests in the ERF study (N5905) and Rotterdam Study (N52480). In short, the g-factor is a general cognitive function phenotype created by principal component analysis of multiple cognitive tests. A higher g-factor is associated with a higher general cognitive function, in contrast to the cognitive measure used for analysis of the ADNI-1 cohort, and the ADAS-Cog13.

The Indiana Memory and Aging Study. The Indiana Memory and Aging Study (IMAS) is an ongoing longitudinal study investigating multimodal neuroimaging, cognition, fluid biomarkers, and genetics in early prodromal stages of AD with follow-up visits every 18 months. IMAS participants included CN participants, euthymic older adults with subjective cognitive decline in the absence of significant psychometric deficits, and patients with amnestic MCI or probable AD. Because of limited sample size compared to other cohorts, analyses were limited to assessment of [¹¹C] Pittsburgh compound B (PiB) positron emission tomography (PET) amyloid status. Thirty-four participants had PET scans to measure brain Aβ load; 30 participants underwent [¹¹C]PiB PET scans on a Siemens HR+ PET scanner; and 4 participants underwent [¹⁸F]Florbetapir PET scans on a Siemens mCT. For the [¹¹C]PiB PET, participants underwent either a 90-minute dynamic scan starting at time of tracer injection or a 50-minute dynamic scan after a 40-minute uptake period after injection of approximately 10 mCi of [¹¹C]PiB. The [¹⁸F] Florbetapir PET scans were collected as a 30-minute dynamic scan after a 40-minute uptake period after an injection of approximately 10 mCi of [¹⁸F]Florbetapir. [¹¹C]PiB and [¹⁸F]Florbetapir scans were motion-corrected and normalized to Montreal Neurologic Institute space using parameters from a same time point structural magnetic resonance imaging (MRI) scan. For the [¹¹C]PiB PET images, a 40- to 90-minute standardized uptake value ratio (SUVR) image was created by averaging the appropriate frames and intensity normalizing to mean cerebellar gray-matter uptake. For the [¹⁸F]Florbetapir PET, a 40- to 70-minute SUVR image was created by averaging the appropriate frames and intensity normalizing to mean whole cerebellar uptake. Finally, amyloid positivity was defined as a mean [¹¹C]PiB PET SURV≥1.37 or a mean [¹⁸F]Florbetapir SURV of ≥1.20 from a cortical grey matter region of interest (ROI). These cutoffs were determined by simultaneous processing of the ADNI [¹¹C]PiB and [¹⁸F]Florbetapir PET images using the same pipeline and adjusting the locally derived cutoffs to best match either the previously reported [¹¹C]PiB PET cutoff of mean cortical SUVR≥1.5 or the [¹⁸F]Florbetapir PET cutoff of SUVR≥1.10, respectively. A side-by-side comparison of the three cohorts, including sample sizes, baseline cognitive diagnoses, and studied outcomes in each cohort, is offered in Table 6.

TABLE 5
Characteristics of IMAS Cohort.

| | CN | MCI | AD |
|---|---|---|---|
| N-subjects | 17 | 10 | 7 |
| Age (years) | 68.4 | 72.1 | 72.4 |
| Women (%) | 76.5 | 60.0 | 71.4 |
| PiB PET + (%) | 29.4% | 60% | 100% |
| MMSE | 29.4 | 28.4 | 24.8 |
| APOE ε4 carriers (%) | 47.1 | 20.0 | 71.4 |

TABLE 6
Sample size, clinical diagnosis and studied
outcomes in each of the included cohorts.

| | ADNI | | Rotterdam | ERF | IMAS | |
|---|---|---|---|---|---|---|
| N | 734 | | 2505 | 905 | 34 | |
| Diagnosis at | 199 | CN | 2505 CN | 905 CN | 17 | CN |
| baseline | 358 | MCI | | | 10 | MCI |
| | 175 | AD | | | 7 | AD |

| | | | | |
|---|---|---|---|---|
| Clinical diagnosis | Yes | No | No | No³ |
| outcome | | | | |
| Cognitive measure | ADAS-Cog13 | g-factor | g-factor | No³ |
| Aβ biomarker | CSF Aβ₁₋₄₂ | No | No | [¹⁸F]Florbetapir PET |
| | | | | [¹¹C]PiB PET |
| MRI measures | SPARE AD¹ | No | No | No |
| | Ventricular | | | |
| | Volume² | | | |

AbsoluteIDQ-p180 kit metabolite measurements. Metabolites were measured with a targeted metabolomics approach using the AbsoluteIDQ-p180 kit (BIOCRATES Life Science AG, Innsbruck, Austria), with an ultra-performance liquid chromatography (UPLC)/MS/MS system [Acquity UPLC (Waters), TQ-S triple quadrupole MS/MS (Waters)] which provides measurements of up to 186 endogenous metabolites quantitatively (amino acids and biogenic amines) and semiquantitatively (acylcarnitines, sphingomyelins, PCs, and lyso-glycerophosphatidylcholines (a 5 acyl) [lysoPCs] across multiple classes). The AbsoluteIDQ-p180 kit has been fully validated according to European Medicine Agency Guidelines on bioanalytical method validation. In addition, plates include an automated technical validation to approve the validity of the run and provide verification of the actual performance of the applied quantitative procedure including instrumental analysis. The technical validation of each analyzed kit plate was performed using MetIDQ software based on results obtained and defined acceptance criteria for blank, zero samples, calibration standards and curves, low/medium/high-level QC samples, and measured signal intensity of internal standards over the plate. This is a highly useful platform that was used in hundreds of publications, including several studies in AD.

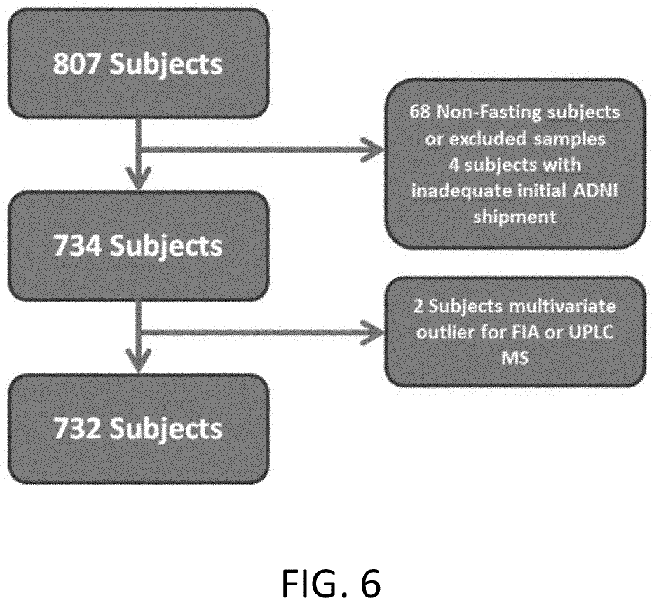

Deidentified samples were analyzed following the manufacturer's protocol, with metabolomics laboratories blinded to diagnosis and pathological data. Serum samples from all 807 ADNI-1 participants were analyzed, but after QC, a smaller number of participants were included in the analysis (FIG. 6). Three participants were excluded because of incomplete clinical data, 70 samples were excluded because of non-fasting status, and two samples were excluded during the multivariate outlier detection step (see the following), leaving 732 participants included in the final analyses. Each assay plate included two sets of replicates: (1) A set of duplicates obtained by pooling the first 72 samples in the study (QC pool duplicates) and (2) 20 blinded analytical duplicates (blinded duplicates).

P180 QC. Metabolites with >40% of measurements below the lower limit of detection (LOD) were excluded from the analysis. Metabolite values were scaled across the different plates using the QC pool duplicates. LOD values were imputed using each metabolite's LOD/2 value. Using the blinded duplicates, we selected metabolites with a coefficient of variation <20% and an intraclass correlation coefficient >0.65. Based on the QC process, 32 of the flow injection analysis metabolites and 14 of the UPLC metabolites were excluded from further analysis (Table 7). We checked for the presence of multivariate outlier participants by evaluating the first and second principal components in each platform. Two multivariate outliers were beyond 7 standard deviations and were there-fore excluded. For the participants with duplicated measurements, we used the average values of the two measured values in further analyses.

TABLE 7
Coefficient of variation (CV) calculated based on replicates
on different plates and result of quality control (QC)
process. CV were not calculated for metabolites with a
high frequency of values below the limit of detection.

| Metabolite | CV interplate | QC result |
|---|---|---|
| Ala | 5.3 | Passed |
| alpha-AAA | 7.2 | Passed |
| Arg | 5.3 | Passed |
| Asn | 4.9 | Passed |
| Asp | 10.4 | Passed |
| C0 | 7.4 | Passed |
| C10 | 6.3 | Passed |
| C10:2 | 8.1 | Passed |
| C12 | 6.9 | Passed |
| C14:1 | 7.3 | Passed |
| C14:1-OH | 6.8 | Passed |
| C14:2 | 7.5 | Passed |
| C16 | 7.2 | Passed |
| C16:1 | 6 | Passed |
| C18 | 5.7 | Passed |
| C18:1 | 6.6 | Passed |
| C18:2 | 4.5 | Passed |
| C2 | 8.9 | Passed |
| C3 | 7.5 | Passed |
| C3-DC (C4-OH) | 12.4 | Passed |
| C4 | 8.1 | Passed |
| C5 | 7.4 | Passed |
| C5-DC (C6-OH) | 12.9 | Passed |
| C6 (C4:1-DC) | 6.8 | Passed |
| C7-DC | 8.3 | Passed |
| C8 | 7.8 | Passed |
| C9 | 8.7 | Passed |
| Cit | 7.5 | Passed |
| Creatinine | 4.1 | Passed |
| Gln | 5.1 | Passed |
| Glu | 9 | Passed |
| Gly | 6.2 | Passed |
| His | 5.7 | Passed |
| Ile | 6.4 | Passed |
| Kynurenine | 8.4 | Passed |
| Lys | 6.6 | Passed |
| lysoPC a C16:0 | 11.1 | Passed |
| lysoPC a C16:1 | 11.2 | Passed |
| lysoPC a C17:0 | 11.3 | Passed |
| lysoPC a C18:0 | 11.1 | Passed |
| lysoPC a C18:1 | 10.6 | Passed |
| lysoPC a C18:2 | 9.6 | Passed |
| lysoPC a C20:3 | 10.5 | Passed |
| lysoPC a C20:4 | 10.2 | Passed |
| lysoPC a C24:0 | 11.8 | Passed |
| lysoPC a C26:0 | 16.6 | Passed |
| lysoPC a C28:0 | 12.4 | Passed |
| lysoPC a C28:1 | 11.5 | Passed |
| Met | 8.1 | Passed |
| Orn | 5.8 | Passed |
| PC aa C28:1 | 6 | Passed |
| PC aa C30:0 | 6.4 | Passed |
| PC aa C32:0 | 6.6 | Passed |
| PC aa C32:1 | 7.9 | Passed |
| PC aa C32:3 | 7.1 | Passed |
| PC aa C34:3 | 6.5 | Passed |
| PC aa C34:4 | 7.5 | Passed |
| PC aa C36:0 | 14.1 | Passed |
| PC aa C36:1 | 8.5 | Passed |
| PC aa C36:5 | 7.9 | Passed |
| PC aa C36:6 | 8 | Passed |
| PC aa C38:0 | 6.8 | Passed |
| PC aa C38:3 | 7.4 | Passed |
| PC aa C38:4 | 6.9 | Passed |
| PC aa C38:5 | 7.8 | Passed |
| PC aa C38:6 | 8.4 | Passed |
| PC aa C40:2 | 11.5 | Passed |
| PC aa C40:3 | 9 | Passed |
| PC aa C40:4 | 7.1 | Passed |
| PC aa C40:5 | 8.1 | Passed |
| PC aa C40:6 | 7.5 | Passed |
| PC aa C42:0 | 7.7 | Passed |
| PC aa C42:1 | 6.8 | Passed |
| PC aa C42:2 | 7.4 | Passed |
| PC aa C42:4 | 9.8 | Passed |
| PC aa C42:5 | 7.7 | Passed |
| PC aa C42:6 | 7.1 | Passed |
| PC ae C30:0 | 5.8 | Passed |
| PC ae C30:2 | 8.5 | Passed |
| PC ae C32:1 | 7 | Passed |
| PC ae C32:2 | 7.3 | Passed |
| PC ae C34:0 | 6.6 | Passed |
| PC ae C34:1 | 7.5 | Passed |
| PC ae C34:2 | 7.4 | Passed |
| PC ae C34:3 | 7.3 | Passed |
| PC ae C36:0 | 6.7 | Passed |
| PC ae C36:1 | 7.2 | Passed |
| PC ae C36:2 | 6.6 | Passed |
| PC ae C36:3 | 7.1 | Passed |
| PC ae C36:4 | 7.3 | Passed |
| PC ae C36:5 | 7.9 | Passed |
| PC ae C38:0 | 6.7 | Passed |
| PC ae C38:3 | 7.7 | Passed |
| PC ae C38:4 | 7.5 | Passed |
| PC ae C38:5 | 7.5 | Passed |
| PC ae C38:6 | 7.6 | Passed |
| PC ae C40:1 | 7.9 | Passed |
| PC ae C40:2 | 7.4 | Passed |
| PC ae C40:3 | 7.3 | Passed |
| PC ae C40:4 | 7.6 | Passed |
| PC ae C40:5 | 7.5 | Passed |
| PC ae C40:6 | 7 | Passed |
| PC ae C42:1 | 7.5 | Passed |
| PC ae C42:2 | 8.6 | Passed |
| PC ae C42:3 | 7.5 | Passed |
| PC ae C42:4 | 8.4 | Passed |
| PC ae C42:5 | 6.9 | Passed |
| PC ae C44:3 | 8.6 | Passed |
| PC ae C44:4 | 6.8 | Passed |
| PC ae C44:5 | 7.7 | Passed |
| PC ae C44:6 | 7.6 | Passed |
| Phe | 6.7 | Passed |
| Pro | 6 | Passed |
| Sarcosine | 12.1 | Passed |
| SDMA | 6.5 | Passed |
| Ser | 6.6 | Passed |
| Serotonin | 12.8 | Passed |
| SM (OH) C14:1 | 7 | Passed |
| SM (OH) C16:1 | 7.4 | Passed |
| SM (OH) C22:1 | 8.2 | Passed |
| SM (OH) C22:2 | 8.1 | Passed |
| SM (OH) C24:1 | 8.4 | Passed |
| SM C16:0 | 7.2 | Passed |
| SM C16:1 | 7.2 | Passed |
| SM C18:0 | 7.6 | Passed |
| SM C18:1 | 7.5 | Passed |
| SM C20:2 | 8 | Passed |
| SM C24:0 | 8.2 | Passed |
| SM C24:1 | 8.7 | Passed |
| SM C26:0 | 10.1 | Passed |
| SM C26:1 | 8.8 | Passed |
| Spermidine | 6.5 | Passed |
| T4-OH-Pro | 5.7 | Passed |
| Taurine | 4 | Passed |
| Thr | 4.3 | Passed |
| Trp | 6.6 | Passed |
| Tyr | 6 | Passed |
| Val | 6.6 | Passed |
| Ac-Orn | NA | Failed |
| ADMA | 12.9 | Failed |
| C10:1 | 7.9 | Failed |
| C12-DC | NA | Failed |
| C12:1 | NA | Failed |
| C14 | 7 | Failed |
| C14:2-OH | 10.2 | Failed |
| C16-OH | 12.3 | Failed |
| C16:1-OH | NA | Failed |
| C16:2 | 9.9 | Failed |
| C16:2-OH | NA | Failed |
| C18:1-OH | NA | Failed |
| C3-OH | 4.9 | Failed |
| C3:1 | 12 | Failed |
| C4-OH-Pro | NA | Failed |
| C4:1 | 69.5 | Failed |
| C5-M-DC | 6.8 | Failed |
| C5-OH (C3-DC-M) | 8.5 | Failed |
| C5:1 | NA | Failed |
| C5:1-DC | 14.8 | Failed |
| C6:1 | 21.3 | Failed |
| Carnosine | NA | Failed |
| DOPA | NA | Failed |
| Dopamine | NA | Failed |
| Histamine | 6 | Failed |
| lysoPC a C14:0 | 2.2 | Failed |
| lysoPC a C26:1 | 19.7 | Failed |
| Met-SO | 23.6 | Failed |
| Nitro-Tyr | NA | Failed |
| PC aa C24:0 | 14.2 | Failed |
| PC aa C26:0 | NA | Failed |
| PC aa C34:1 | 11.3 | Failed |
| PC aa C34:2 | 11.8 | Failed |
| PC aa C36:2 | 10.3 | Failed |
| PC aa C36:3 | 7.4 | Failed |
| PC aa C36:4 | 9.1 | Failed |
| PC aa C40:1 | 8.2 | Failed |
| PC ae C30:1 | 39 | Failed |
| PC ae C38:1 | 49 | Failed |
| PC ae C38:2 | 10.4 | Failed |
| PC ae C42:0 | NA | Failed |
| PEA | NA | Failed |
| Putrescine | 30.3 | Failed |
| Spermine | 89.2 | Failed |

CSF Aβ₁₋₄₂ and tau biomarkers. Lumbar puncture was performed in the mornings after an overnight fast. Aβ₁₋₄₂, total tau (t-tau), and tau phosphorylated at threonine 181 (p-tau181) were measured using the multiplex xMAP Luminex platform (Luminex Corp, Austin, Tex.) with Innogenetics immunoassay kit-based reagents (INNO-BIA AlzBio3; Ghent, Belgium; for research use-only reagents). CSF samples were available and measured for 48.8% of the CN, 52% of the MCI, and 54.9% of the AD participants. Aβ₁₋₄₂-defined groups were classified as normal or pathological based on the previously published concentration (192 pg/mL).

MRI measures. A 1.5-T MRI non-accelerated sagittal volumetric 3D magnetization-prepared rapid gradient-echo MRI images were acquired at each performance site for the ADNI-1 participants (adni-info.org; adni.loni.usc.edu). Only images that passed QC evaluations were included. Cortical gray-matter volumes were processed using the FreeSurfer version 4.4 image processing framework (surfer.nmr.mgh.harvard.edu). FreeSurfer ventricular volume of MRI scans that passed the QC was adjusted for total intracranial volume and used for longitudinal analyses. The Spatial Pattern of Abnormality for Recognition of Early Alzheimer's Disease (SPARE-AD), an index that captures brain atrophy related to AD and has shown association with AD CSF biomarker and clinical measures, and was calculated for the baseline visit of ADNI-1 participants, was assessed in the present analysis.

Medication adjustment. In the ADNI and IMAS cohort, 41 major medication classes used to treat psychiatric (including different categories of benzodiazepines, antipsychotics, and antidepressants) and cardiovascular conditions (including different categories of antihypertensives, cholesterol treatment, and antidiabetics), as well as dietary supplements (Co-Q10, fish oil, nicotinic acid, and acetyl L-carnitine), were systematically coded and available for model-based evaluations of the influence of each drug type on metabolite levels. Intake of any medication within a category was coded as present or absent. Dose effect was not evaluated. The list of the studied medication categories and the percentage of subjects taking these medications in each of the diagnostic categories for the ADNI cohort is listed in Table 8.

Statistical analysis. Metabolites with a skewness >2 that showed a departure of the normality distribution (D'Agostino test P-value <0.05) were log 10 transformed to normalize their distribution. A two-stage regression approach was implemented, whereby metabolites were first adjusted for confounding medications and dietary supplements in a linear regression model. For each metabolite, medications were backward-selected via Bayesian information criteria to select an optimal combination of medications for preventing confounding while limiting model complexity. The residuals for each metabolite were then carried forward to test associations with clinical outcomes.

Sample Preparation. Samples were prepared using the AbsoluteIDQ® p180 kit (Biocrates Life Sciences AG, Innsbruck, Austria) in strict accordance with the user manual. In brief, after the addition of 10 μL of the supplied internal standard solution to each well on a filter spot of the 96-well extraction plate, 10 μL of each serum sample, low/medium/high quality control (QC) samples, blank, zero sample, or calibration standard were added to the appropriate wells. The plate was then dried under a gentle stream of nitrogen. The samples were derivatized with phenyl isothiocyanate (PITC) for the amino acids and biogenic amines. Sample extract elution is performed with 5 mM ammonium acetate in methanol. Furthermore, sample extracts were diluted with either 40% methanol in water for the UPLC-MS/MS analysis (15:1) or kit running solvent (Biocrates Life Sciences AG) for flow injection analysis (FIA)-MS/MS (20:1).

Quality Control Samples. The analysis of the samples using the AbsoluteIDQ® p180 kit was performed using four specific sets of quality controls. First, low/mid/high level QC samples provided by Biocrates Life Sciences AG were prepared and analyzed on each plate as recommended by the manufacturer. These QC samples were used for a technical validation of each kit plate. Second, the NIST standard reference material (SRM)-1950 reference plasma was prepared and analyzed three times on each kit plate in order to measure intra- and inter-assay reproducibility, although this was not used for data curation because of differences in levels between plasma and serum. Third, to allow appropriate inter-plate abundance scaling based specifically on this cohort of samples, we generated a Study Pool QC by combining approximately 10 μL from the first 76 samples for analysis. This sample was frozen in aliquots of an appropriate volume and analyzed independently on all of the plates analyzed in this study. The pooled sample was prepared and analyzed twice on each plate, once before and once after the study samples.

Quantitative UPLC-MS/MS and FIA-MS/MS Analysis. Sample analysis was performed based on Standard Operating Procedures provided by Biocrates for the AbsoluteIDQ® p180 kit. Chromatographic separation of amino acids and biogenic amines was performed using a ACQUITY UPLC System (Waters Corporation) using a ACQUITY 2.1 mm×50 mm 1.7 μm BEH C18 column fitted with a ACQUITY BEH C18 1.7 μm VanGuard guard column, and quantified by calibration curve using a linear regression with 1/x weighting. Acylcarnitines, sphingolipids, and glycerophospholipids, were analyzed by flow injection analysis tandem mass spectrometry (FIA-MS/MS), quantified by internal standard calibration. Thus, FIA-MS/MS analytes are reported as semi-quantitative values except where a stable-isotope labeled internal standard of that exact analyte was used. Samples for both UPLC and FIA were introduced directly into a Xevo TQ-S mass spectrometer (Waters Corporation) using positive electrospray ionization operating in the Multiple Reaction Monitoring (MRM) mode. MRM transitions (compound-specific precursor to product ion transitions) for each analyte and internal standard were collected over the appropriate retention time using tune files and acquisition methods provided in the AbsoluteIDQ® p180 kit. The UPLC data were imported into TargetLynx (Waters Corporation) for peak integration, calibration and concentration calculations. The UPLC data from TargetLynx and FIA data were analyzed using Biocrates' MetIDQ software.

Accounting for effects of medications in ADNI. Medication information, including dosage and reason for taking, was collected from research participants in the form of free text. In order to account for exposure to specific drugs and drug classes in our analysis, it was necessary to convert that unstructured data into structured terms representing both the specific drug and the drug class[es] to which it belonged. We used the RxNorm API (application programming interface) to convert drug names, including synonyms and misspellings, into coded drug terms from RxNorm, a standardized drug terminology developed and maintained by the National Library of Medicine (NLM). Inexact matches were manually reviewed and corrected where necessary. The RxNorm API was then used to identify corresponding drug classes for the respective coded medications. For example, citalopram, citalopran, citalporam, and Celexa all mapped to the concept “citalopram” with RXCUI (RxNorm Concept Unique Identifier) “2556”. Along with drugs like Zoloft, Lexapro, and Prozac, they were mapped to the classes “Antidepressive Agents, Second-Generation” and “Serotonin Reuptake Inhibitor.” An iterative approach was used to identify drug classes of interest from hundreds of partially overlapping possible classifications. Drug classes were identified for a core set of medications. The other medications sharing these classes were determined and all their respective drug classes were identified, further generating new medications and classes. Through iteration and pruning based on review with clinical experts, the final set of ontology classes of interest was created. Statistical approaches accounting for effect of medication on metabolites measured can be found in statistical method section.

AD medications (anti-cholinesterases) are a special issue in this context. As these medications are taken only by AD cases (about 90%) and advanced MCI subjects (about 40%) but not by controls, this medication class largely coincides with diagnosis leading to a highly significant correlation between medication status and diagnosis. As mentioned, we intentionally excluded diagnosis as covariate in regression analyses because the investigated clinical variables naturally also show high levels of correlation with diagnosis (and, thus, also with these medications). In order to find out if anti-cholinesterases significantly alter the effect of metabolites on AD-related clinical variables, we performed regression analyses for all significant associations reported in our study in MCI subjects stratified by AD medication status (202 non-takers vs. 157 takers) and then investigated if metabolite regression coefficients are significantly different between these groups using the method described by Paternoster et al. Interestingly, although three of the four clinical variables (only non-significant was for t-tau/Aβ₁₋₄₂ ratio) are significantly correlated with AD medication status, metabolite effect sizes did not differ significantly between takers and non-takers in most cases. This indicates that the reported findings are most probably no artifacts resulting from excluding AD medications from the association analyses.

CSF collection and Aβ₁₋₄₂ measurement. CSF was collected into polypropylene collection tubes or syringes provided to each site, transferred into polypropylene transfer tubes without any centrifugation step followed by freezing on dry ice within 1 hr after collection, and overnight shipment to the ADNI Biomarker Core laboratory at the University of Pennsylvania Medical Center on dry ice. The samples were thawed for 1 hour at room temperature, gently mixed and divided into aliquots (0.5 ml). The aliquots were stored in bar code-labeled polypropylene vials at −80° C. The analyte-specific detection antibodies were HT7, for tau, and 3D6, for the N-terminus of Aβ.

Longitudinal analyses using mixed-effects models. For evaluation of longitudinal associations between metabolites between ventricular volume and ADAS-Cog13, a linear mixed-effects model was used including the respective covariates as well as the metabolite plus the interaction term for time*metabolite as fixed effects and time grouped by samples as random effects. To enhance the power of these analyses, we transformed the response variables (square root of raw ADAS-Cog13 scores and a Box-Cox transformation of ventricular volume then normalized to intracranial volume) to approximately follow a normal distribution.

The cross-sectional association with categorical outcomes (clinical diagnosis and CSF Aβ₁₋₄₂ group) was studied using a logistic regression model. For the cross-sectional quantitative outcomes (Mau/Aβ₁₋₄₂ ratio, SPARE-AD, and ADAS-Cog13), a linear regression model was applied. Age and gender were forced covariates in all the models associating with clinical variables, and education was also forced into the models for ADAS-Cog13 and clinical diagnosis, whereas APOE ε4 was backward-selected based on Bayesian information criteria for each outcome (Table 9). Diagnosis was not included as a covariate in the models in the primary analyses that studied Aβ₁₋₄₂, Mau/Aβ₁₋₄₂ ratio, SPARE-AD, and ADAS-Cog13 associations. The P-values were Bonferroni corrected to adjust for multiple comparisons and a corrected 0.05 two-tailed P-value was considered significant. A Cox hazard model including age, gender, APOE ε4 presence, and education as covariates was used to evaluate the association of metabolite levels with progression from MCI to AD with a median follow-up of 3.0 years (interquartile range [IQR]: 2.0-6.1). A mixed-effects model that included age, gender, education, APOE ε4 presence, time, and metabolite level as independent variables was used to study longitudinal associations between the metabolites and volumetric MRI changes (transformed to normalized distribution) during follow-up in the MCI participants (AD participants were excluded because of short follow-up). A mixed-effects model was also used to evaluate the association of metabolites with change in ADAS-Cog13 (transformed to normalized distribution) and included education as an additional covariate. Both models accounted for baseline cognitive and MRI measures for each participant. Median follow-up times for the MRI and cognitive analyses were 3.0 years (IQR: 2.0-5.0). An interaction with time was included in all mixed-effects models for the studied metabolites.

TABLE 9
Covariate Selection for Association of Metabolites with Clinical Outcomes.

| | | | Forced | Selectable | Selected | |
|---|---|---|---|---|---|---|
| Outcome | Model | N | Covariates | Covariates | Covariates | Final Model |
| AD vs CN | Logistic | 374 | Age, | APOE ε4 | APOE ε4 | Age + Gender + |
| | regression | | Gender | | | Education + APOE |
| | | | | | | ε4 + Metabolite |
| | | | | | | Residuals |
| MCI vs CN | Logistic | 560 | Age, | APOE ε4 | APOE ε4 | Age + Gender + |
| | regression | | Gender | | | APOE4 + Metabolite |
| | | | | | | Residuals |
| Aβ₁₋₄₂ | Logistic | 379 | Age, | APOE ε4 | APOE ε4 | Age + Gender + APOE |
| | regression | | Gender | | | ε4 + Metabolite |
| | | | | | | Residuals |
| SPARE-AD | Linear | 733 | Age, | APOE ε4 | none | Age + Gender + |
| | regression | | Gender | | | Metabolite |
| | | | | | | Residuals |
| ADAS-Cog13 | Linear | 727 | Age, | APOE ε4 | none | Age + Gender + |
| | regression | | Gender, | | | Education + |
| | | | Education | | | Metabolite |
| | | | | | | Residuals |
| T-tau/Aβ₁₋₄₂ | Linear | 375 | Age, | APOE ε4 | none | Age + Gender + |
| ratio | regression | | Gender | | | Metabolite |
| | | | | | | Residuals |

In the Rotterdam study, a linear regression model was fitted for the cross-sectional analysis with g-factor as the outcome and valine as the determinant, adjusting for age, gender, lipid-lowering medication, and education. P-values and effect estimates of the significant metabolites are reported. [¹¹C]PiB PET analysis for IMAS samples included age, gender, and APOE ε4 presence, along with the Aβ₁₋₄₂ status on PET, as independent predictors of target metabolite measures using a linear regression model. All analyses were performed using the R software package.

Co-expression network construction and module analysis. The global baseline cross-sectional correlation structure of metabolites was investigated and their correlation with a subset of clinical and biomarker measures at baseline (Aβ₁₋₄₂, tau/Aβ₁₋₄₂ ratio, and ADAS-Cog13). The p180 coexpression network was built based on baseline-normalized data adjusted for age, education, gender, and APOE ε4 presence using the WGCNA R package.

Partial correlation analysis. Biochemically related metabolites and propagation patterns of effects on the clinical variables were investigated from a network perspective. A Gaussian graphical model (GGM) calculation was performed using the GeneNet R package with default parameters. To illustrate effect propagation on clinical variables, we colored the resulting network. In brief, a GGM is an undirected graphical model based on partial correlation coefficients, that is, pairwise correlation coefficients conditioned against correlations with all other included variables. GGMs, contrary to correlation networks, thus can reveal the direct relations between metabolites. To account for correlations between metabolites and clinical or other potentially predictive variables, we used metabolite residuals that accounted for effects of medication and dietary supplements (as described previously) and additionally included age, gender, APOE ε4 presence, and education as covariates in the GGM generation process. To obtain significant partial correlations, we used a significance threshold of 0.05 after Bonferroni correction for all possible edges in the model (0.05/10,296=4.86×10⁻⁶). For each clinical variable, we colored the network representation of the GGM using the results of our regression analyses using sign(β)*(−log 10(P)) to visualize both strength of association and direction of effect.

TABLE 11
Analysis adjusted by clinical diagnosis.

| | ADAScog13 | | SPARE-AD | | Aβ₁₋₄₂ Group | |
|---|---|---|---|---|---|---|
| | | Bonferroni | | Bonferroni | | Bonferroni |
| | Coef. | p-value | Coef. | p-value | Coef. | p-value |
| C12 | 2.7167 | 1.0000 | −0.0020 | 1.0000 | 0.7540 | 1.0000 |
| C14:1 | 26.4082 | 0.8558 | 0.2662 | 1.0000 | 10.8108 | 1.0000 |
| C16:1 | 4.8041 | 0.2091 | 1.8965 | 1.0000 | 1.0163 | 1.0000 |
| C18 | 22.1538 | 1.0000 | 0.3719 | 1.0000 | 13.8559 | 1.0000 |
| PC ae C36:2 | 0.0684 | 1.0000 | 1.8405 | 1.0000 | 0.1435 | 0.1026 |
| PC ae C40:3 | 0.3192 | 1.0000 | −0.0027 | 1.0000 | 5.1139 | 0.1559 |
| PC ae C42:4 | 0.8493 | 1.0000 | −0.1617 | 1.0000 | 2.1384 | 0.1071 |
| PC ae C44:4 | 5.4702 | 1.0000 | 0.2460 | 1.0000 | 5.6136 | 0.1064 |
| SM (OH) C14:1 | 0.1511 | 1.0000 | 0.9965 | 0.5009 | 0.2199 | 0.2544 |
| SM C16:0 | 0.0110 | 1.0000 | 0.0082 | 1.0000 | 0.0131 | 0.1402 |
| SM C20:2 | 2.4653 | 0.0342 | 0.0014 | 1.0000 | 0.4956 | 1.0000 |
| alpha-AAA | −1.8425 | 0.1208 | 0.0214 | 1.0000 | −0.5331 | 1.0000 |
| Val | −0.0105 | 0.5751 | −0.2733 | 0.0237 | −0.0020 | 1.0000 |

Regression and Bonferroni corrected p-values.

TABLE 12
Analysis of outcomes in each of the clinical group.

| | ADAS-Cog13 | | | SPARE-AD | | | Aβ₁₋₄₂ Group | | |
|---|---|---|---|---|---|---|---|---|---|
| | CN | MCI | AD | CN | MCI | AD | CN | MCI | AD |
| alpha-AAA | −0.857 | −1.033 | −4.942 | −0.163 | −0.291 | −0.308 | 0.417 | 0.971 | −1.233 |
| | (0.3807) | (0.4492) | (0.0685) | (0.3976) | (0.0756) | (0.2229) | (0.4039) | (0.0571) | (0.3105) |
| C12 | 4.751 | 7.337 | 4.594 | 0.084 | 0.196 | 0.347 | 1.076 | −1.447 | −0.675 |
| | (0.0445) | (0.0034) | (0.3215) | (0.8594) | (0.5218) | (0.4189) | (0.3693) | (0.1225) | (0.772) |
| C14:1 | 4.954 | 11.128 | 7.818 | 0.273 | 0.263 | 0.277 | 2.32 | −2.935 | −0.576 |
| | (0.1199) | (5e−04) | (0.1813) | (0.6664) | (0.5006) | (0.6078) | (0.1616) | (0.024) | (0.8475) |
| C16:1 | 85.279 | 77.586 | 50.673 | −0.759 | 0.54 | 0.414 | −4.964 | −22.436 | −21.697 |
| | (0.01) | (0.0079) | (0.5252) | (0.9092) | (0.8783) | (0.9554) | (0.7675) | (0.0524) | (0.5492) |
| C18 | 0.162 | 0.442 | 0.183 | −0.048 | 0.025 | −0.003 | −0.05 | −0.176 | −0.26 |
| | (0.1905) | (0.001) | (0.468) | (0.0475) | (0.1269) | (0.9143) | (0.4239) | (0.0045) | (0.0501) |
| PC ae | 4.291 | 9.038 | 12.966 | −1.866 | 1.453 | −0.063 | −2.645 | −5.703 | −10.566 |
| C36:2 | (0.3813) | (0.0538) | (0.1851) | (0.0519) | (0.0101) | (0.945) | (0.2891) | (0.0059) | (0.0555) |
| PC ae | 3.831 | 0.774 | 3.504 | −0.256 | 0.778 | 0.034 | −2.373 | −2.005 | 0.437 |
| C40:3 | (0.0301) | (0.7046) | (0.3712) | (0.4682) | (0.0014) | (0.9242) | (0.0122) | (0.0241) | (0.8123) |
| PC ae | 9.639 | 5.329 | 17.301 | −0.458 | 2.015 | −0.242 | −5.725 | −6.241 | −2.61 |
| C42:4 | (0.0532) | (0.2802) | (0.0863) | (0.6448) | (6e−04) | (0.7954) | (0.0291) | (0.0075) | (0.6277) |
| PC ae | 0.027 | 0.023 | 0.03 | 0 | 0.002 | 0 | −0.018 | −0.007 | −0.027 |
| C44:4 | (0.0192) | (0.0705) | (0.267) | (0.855) | (0.1239) | (0.9509) | (0.0075) | (0.1372) | (0.0615) |
| SM (OH) | 0.247 | 3.528 | 4.03 | −0.218 | 0.183 | 0.256 | 1.114 | −0.887 | −3.264 |
| C14:1 | (0.8639) | (0.0134) | (0.154) | (0.4426) | (0.2931) | (0.3258) | (0.1434) | (0.1198) | (0.0549) |
| SM C16:0 | 0.252 | 0.582 | 0.561 | −0.076 | 0.052 | −0.022 | −0.259 | −0.143 | −0.49 |
| | (0.2547) | (0.0103) | (0.225) | (0.0822) | (0.0583) | (0.6115) | (0.0334) | (0.1049) | (0.0667) |
| SM C20:2 | 22.141 | 56.658 | 45.517 | −0.103 | 2.808 | 0.777 | 13.823 | −21.788 | −12.846 |
| | (0.364) | (0.0027) | (0.1875) | (0.983) | (0.2241) | (0.8085) | (0.2912) | (0.0223) | (0.5541) |
| Val | 0.006 | 0.002 | −0.018 | 0 | −0.001 | 0.001 | 0.003 | 0.001 | −0.009 |
| | (0.395) | (0.8357) | (0.325) | (0.7408) | (0.6149) | (0.4076) | (0.4895) | (0.7499) | (0.325) |

Regression coefficient (Non-Bonferroni corrected p-value).

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses, as well as the following claims:

Clause 1. A method of diagnosing or detecting Alzheimer's disease in a subject comprising obtaining a sample from a subject and performing biochemical analysis on the sample to detect the presence of at least one biomarker metabolite, wherein the at least one biomarker metabolite is selected from the group consisting of a carnitine biomarker metabolite, a phosphatidylcholine biomarker metabolite, a sphingomyelin biomarker metabolite, and combinations thereof; wherein detection of the at least one biomarker metabolite is associated with the subject having at least one independent indicator of Alzheimer's disease; and wherein the subject is diagnosed with having Alzheimer's disease, or an increased risk of Alzheimer's disease, if at least one biomarker metabolite is detected.

Clause 2. The method of clause 1, wherein the sample from the subject is whole blood, serum, plasma, or cerebral spinal fluid (CSF).

Clause 3. The method of clause 1 or clause 2, wherein the carnitine biomarker metabolite is at least one of Dodecanoyl-L-carnitine (C12), Tetradecenoyl-L-carnitine (C14:1), Hexadecenoyl-L-carnitine (C16:1), Octadecanoyl-L-carnitine (C18), or combinations thereof.

Clause 4. The method of any of clauses 1-3, wherein the phosphatidylcholine biomarker metabolite is at least one of Phosphatidylcholine acyl-alkyl C36:2 (PC ae C36:2), Phosphatidylcholine acyl-alkyl C40:3 (PC ae C40:3), Phosphatidylcholine acyl-alkyl C42:4 (PC ae C42:4), Phosphatidylcholine acyl-alkyl C44:4 (PC ae C44:4), or combinations thereof.

Clause 5. The method of any of clauses 1-4, wherein the sphingomyelin biomarker metabolite is at least one of Hydroxysphingomyelin C14:1 (SM (OH) C14:1), Sphingomyelin C16:0 (SM C16:0), Sphingomyelin C20:2 (SM C20:2), or combinations thereof.

Clause 6. The method of any of clauses 1-5, wherein if the concentration of the at least one biomarker metabolite in the sample from the subject is higher than the concentration of the at least one biomarker in a control sample, the subject is diagnosed with having at least one independent indicator of Alzheimer's disease.

Clause 7. The method of clause 6, wherein the control sample is taken from a subject or population of subjects with normal cognition.

Clause 8. The method of any of clauses 1-7, further comprising detecting at least one negatively correlated biomarker metabolite, wherein detecting the at least one negatively correlated biomarker metabolite is associated with an absence of at least one independent indicator of Alzheimer's disease.

Clause 9. The method of any of clauses 1-8, wherein the negatively correlated biomarker metabolite is at least one of valine and α-aminoadipic acid, or combinations thereof.

Clause 10. The method of any of clauses 1-9, wherein if the concentration of the at least one negatively correlated biomarker metabolite in the sample from the subject is higher than the concentration of the at least one negatively correlated biomarker metabolite in a control sample, the subject is diagnosed with not having at least one independent indicator of Alzheimer's disease.

Clause 11. The method of any of clauses 1-10, wherein at least one independent indicator of Alzheimer's disease comprises at least one of an increase in Alzheimer's Disease Assessment Scale cognitive subscale 13 (ADAS-Cog 13) score, an increase in Spatial Pattern of Abnormality for Recognition of Early Alzheimer's disease (SPARE-AD) score, an increase in brain ventricular volume, presence of Amyloid β₁₋₄₂ protein fragment (Aβ₁₋₄₂), an increased total Tau (T-tau)/Aβ₁₋₄₂ ratio, or combinations thereof.

Clause 12. The method of any of clauses 1-11, wherein the detection of at least one of PC ae C36:2, PC ae C40:3, PC ae C42:4, PC ae C44:4, SM (OH) C14:1, SM C16:0, or combinations thereof indicates the subject has at least one independent indicator of Alzheimer's disease comprising the presence of Aβ₁₋₄₂.

Clause 13. The method of any of clauses 1-12, wherein the detection of at least one of C18, PC ae C36:2, SM C16:0, SM C20:2, or combinations thereof indicates that the subject has at least one independent indicator of Alzheimer's disease comprising an increased total Tau (T-tau)/Aβ₁₋₄₂ ratio.

Clause 14. The method of any of clauses 1-13, wherein the detection of at least of C14:1, C16:1, SM C20:2, or combinations thereof indicates that the subject has at least one independent indicator of Alzheimer's disease comprising an increase in ADAS-Cog 13 score.

Clause 15. The method of any of clauses 1-14, wherein the detection of at least one of C12, C16:1, PC ae C42:4, PC ae C44:4, or combinations thereof indicates that the subject has at least one independent indicator of Alzheimer's disease comprising an increase in SPARE-AD score.

Clause 16. The method of any of clauses 1-15, wherein the detection of at least one of PC ae C40:3, PC ae C42:4, PC ae C44:4, SM (OH) C14:1, SM C16:0, SM C20:2, or combinations thereof indicates that the subject has at least one independent indicator of Alzheimer's disease comprising one or more of an increase in ADAS-Cog 13 score, and an increase in brain ventricular volume.

Clause 17. The method of any of clauses 1-16, further comprising initiating treatment for Alzheimer's disease in the subject diagnosed with Alzheimer's disease.

Clause 18. A method of diagnosing or detecting Mild Cognitive Impairment (MCI) in a subject comprising obtaining a sample from a subject and performing biochemical analysis on the sample to detect the presence of at least one biomarker metabolite, wherein the at least one biomarker metabolite is selected from the group consisting of a carnitine biomarker metabolite, a phosphatidylcholine biomarker metabolite, a sphingomyelin biomarker metabolite, and combinations thereof; wherein detection of the at least one biomarker metabolite is associated with the subject having at least one independent indicator of MCI; and wherein the subject is diagnosed with having MCI, or an increased risk of MCI, if at least one biomarker metabolite is detected.

Clause 19. The method of clause 18, further comprising initiating treatment for MCI in the subject diagnosed with MCI.

Clause 20. A method of predicting the outcome of a subject suspected of having Alzheimer's disease comprising obtaining a sample from a subject; performing biochemical analysis on the sample to detect the presence of at least one biomarker metabolite, wherein the at least one biomarker metabolite is selected from the group consisting of a carnitine biomarker metabolite, a phosphatidylcholine biomarker metabolite, a sphingomyelin biomarker metabolite, and combinations thereof; and assessing at least one independent indicator of Alzheimer's disease in the subject; wherein detection of the at least one biomarker metabolite is associated with the subject having at least one independent indicator of Alzheimer's disease; and wherein the subject is predicted to develop Alzheimer's disease if at least one biomarker metabolite is detected.

Clause 21. The method of clause 20, further comprising initiating treatment for Alzheimer's disease in the subject predicted to develop Alzheimer's disease.

1. A method for preparing and analyzing a sample containing a biomarker metabolite useful for the analysis and identification of metabolic changes associated with Alzheimer's disease in a subject, the method comprising:
obtaining a sample from a subject; and
performing biochemical analysis on the sample to detect the presence of at least one biomarker metabolite, wherein the at least one biomarker metabolite is selected from the group consisting of a carnitine biomarker metabolite, a phosphatidylcholine biomarker metabolite, a sphingomyelin biomarker metabolite, and combinations thereof;
wherein detection of the at least one biomarker metabolite is associated with the subject having at least one independent indicator of Alzheimer's disease; and
wherein the subject is diagnosed with having Alzheimer's disease, or an increased risk of Alzheimer's disease, if at least one biomarker metabolite is detected.

2. The method of claim 1, wherein the sample from the subject is whole blood, serum, plasma, or cerebral spinal fluid (CSF).

3. The method of claim 1, wherein the camitine biomarker metabolite is at least one of Dodecanoyl-L-carnitine (C12), Tetradecenoyl-L-carnitine (C14:1), Hexadecenoyl-L-carnitine (C16:1), Octadecanoyl-L-carnitine (C18), or combinations thereof.

4. The method of claim 1, wherein the phosphatidylcholine biomarker metabolite is at least one of Phosphatidylcholine acyl-alkyl C36:2 (PC ae C36:2), Phosphatidylcholine acyl-alkyl C40:3 (PC ae C40:3), Phosphatidylcholine acyl-alkyl C42:4 (PC ae C42:4), Phosphatidylcholine acyl-alkyl C44:4 (PC ae C44:4), or combinations thereof.

5. The method of claim 1, wherein the sphingomyelin biomarker metabolite is at least one of Hydroxysphingomyelin C14:1 (SM (OH) C14:1), Sphingomyelin C16:0 (SM C16:0), Sphingomyelin C20:2 (SM C20:2), or combinations thereof.

6. The method of claim 1, wherein if the concentration of the at least one biomarker metabolite in the sample from the subject is higher than the concentration of the at least one biomarker in a control sample, the subject is diagnosed with having at least one independent indicator of Alzheimer's disease.

7. The method of claim 6, wherein the control sample is taken from a subject or population of subjects with normal cognition.

8. The method of claim 1, further comprising detecting at least one negatively correlated biomarker metabolite, wherein detecting the at least one negatively correlated biomarker metabolite is associated with an absence of at least one independent indicator of Alzheimer's disease.

9. The method of claim 1, wherein the negatively correlated biomarker metabolite is at least one of valine and α-aminoadipic acid, or combinations thereof.

10. The method of claim 1, wherein if the concentration of the at least one negatively correlated biomarker metabolite in the sample from the subject is higher than the concentration of the at least one negatively correlated biomarker metabolite in a control sample, the subject is diagnosed with not having at least one independent indicator of Alzheimer's disease.

11. The method of claim 1, wherein at least one independent indicator of Alzheimer's disease comprises at least one of an increase in Alzheimer's Disease Assessment Scale cognitive subscale 13 (ADAS-Cog 13) score, an increase in Spatial Pattern of Abnormality for Recognition of Early Alzheimer's disease (SPARE-AD) score, an increase in brain ventricular volume, presence of Amyloid β 1-42 protein fragment (Aβ1-42), an increased total Tau (T-tau)/Aβ1-42 ratio, or combinations thereof.

12. The method of claim 1, wherein the detection of at least one of PC ae C36:2, PC ae C40:3, PC ae C42:4, PC ae C44:4, SM (OH) C14:1, SM C16:0, or combinations thereof indicates the subject has at least one independent indicator of Alzheimer's disease comprising the presence of Aβ1-42.

13. The method of claim 1, wherein the detection of at least one of C18, PC ae C36:2, SM C16:0, SM C20:2, or combinations thereof indicates that the subject has at least one independent indicator of Alzheimer's disease comprising an increased total Tau (T-tau)/Aβ1-42 ratio.

14. The method of claim 1, wherein the detection of at least of C14:1, C16:1, SM C20:2, or combinations thereof indicates that the subject has at least one independent indicator of Alzheimer's disease comprising an increase in ADAS-Cog 13 score.

15. The method of claim 1, wherein the detection of at least one of C12, C16:1, PC ae C42:4, PC ae C44:4, or combinations thereof indicates that the subject has at least one independent indicator of Alzheimer's disease comprising an increase in SPARE-AD score.

16. The method of claim 1, wherein the detection of at least one of PC ae C40:3, PC ae C42:4, PC ae C44:4, SM (OH) C14:1, SM C16:0, SM C20:2, or combinations thereof indicates that the subject has at least one independent indicator of Alzheimer's disease comprising one or more of an increase in ADAS-Cog 13 score, and an increase in brain ventricular volume.

17. The method of claim 1, further comprising initiating treatment for Alzheimer's disease in the subject diagnosed with Alzheimer's disease.

18. A method for preparing and analyzing a sample containing a biomarker metabolite useful for the analysis and identification of metabolic changes associated with Mild Cognitive Impairment (MCI) in a subject,
the method comprising:
obtaining a sample from a subject; and
performing biochemical analysis on the sample to detect the presence of at least one biomarker metabolite, wherein the at least one biomarker metabolite is selected from the group consisting of a carnitine biomarker metabolite, a phosphatidylcholine biomarker metabolite, a sphingomyelin biomarker metabolite, and combinations thereof;
wherein detection of the at least one biomarker metabolite is associated with the subject having at least one independent indicator of MCI; and
wherein the subject is diagnosed with having MCI, or an increased risk of MCI, if at least one biomarker metabolite is detected.

19. The method of claim 18, further comprising initiating treatment for MCI in the subject diagnosed with MCI.

20. A method for preparing and analyzing a sample containing a biomarker metabolite useful for predicting the outcome of a subject suspected of having Alzheimer's disease, the method comprising:
obtaining a sample from a subject;
performing biochemical analysis on the sample to detect the presence of at least one biomarker metabolite, wherein the at least one biomarker metabolite is selected from the group consisting of a carnitine biomarker metabolite, a phosphatidylcholine biomarker metabolite, a sphingomyelin biomarker metabolite, and combinations thereof; and
assessing at least one independent indicator of Alzheimer's disease in the subject;
wherein detection of the at least one biomarker metabolite is associated with the subject having at least one independent indicator of Alzheimer's disease; and
wherein the subject is predicted to develop Alzheimer's disease if at least one biomarker metabolite is detected.

21. The method of claim 20, further comprising initiating treatment for Alzheimer's disease in the subject predicted to develop Alzheimer's disease.
